(19) United States
(12) Patent Application Publication
(10) Pub. No.: US 2006/0280812 A1
(43) Pub. Date: Dec. 14, 2006

(54) Compositions and methods for the treatment of muscular dystrophy

(76) Inventors: C. George Carlson, Kirksville, MO (US); Abbas Samadi, Kirksville, MO (US)

Correspondence Address: LATHROP & GAGE LC, 4845 PEARL EAST CIRCLE, SUITE 300, BOULDER, CO, 80301, US

(21) Appl. No.: 11/439,714

(22) Filed: May 24, 2006

Related U.S. Application Data
(60) Provisional application No. 60/684,504, filed on May 24, 2005.
(60) Provisional application No. 60/762,394, filed on Jan. 26, 2006.

(51) Int. Cl. A61K 36/185; A61K 38/08; A61K 31/7034; A61K 31/522; A61K 31/517; A61K 31/513; A61K 31/505; A61K 48/00; A61K 31/426; A61K 36/78

(52) U.S. Cl. 424/725; 424/729; 514/17; 514/169; 514/22; 514/303; 514/266.1; 514/275; 514/277; 514/312; 514/263.31; 514/44; 514/310; 514/269; 514/369; 514/352; 514/357; 514/562; 514/291; 514/394; 514/419; 514/690

(57) ABSTRACT

Compositions and methods for treatment of individuals diagnosed with a dystrophin deficiency are disclosed. In particular, inhibitors of NFκB activation, such as pyrrolidine dithiocarbamate (PDTC), have been shown to prevent and reverse muscle damage in animals lacking dystrophin. Such compositions and methods are useful in the treatment of individuals with muscular dystrophy.

### RELATED APPLICATIONS

This application claims priority of U. S. Provisional Application No. 60/684,504, filed May 24, 2005, and U. S. Provisional Application No. 60/762,394, filed Jan. 26, 2006, the content of which is hereby incorporated into this application by reference.

### BACKGROUND

1. Field of the Invention

The present invention relates to pharmaceutical compositions and methods for the treatment of muscular dystrophies.

2. Description of the Related Art

Muscular dystrophies (MD) are a group of genetic diseases that afflict more than 50,000 Americans. The diseases are characterized by progressive weakness and degeneration of the skeletal muscle fibers that control movement. Both voluntary and involuntary muscles, such as heart and respiratory muscles, are replaced by fat and connective tissue in the late stages of the disease. Muscular dystrophies are a heterogeneous disorders.

Muscular dystrophies are heterogeneous in that the causes of the disorders are diverse. One of the most common forms of muscular dystrophy is Duchenne muscular dystrophy (DMD), which afflicts about 1 out of every 3500 males. DMD is characterized by a near complete lack of dystrophin production, which is typically caused by mutations in the gene coding for the dystrophin protein. While some females may carry the mutations without showing symptoms of the disease, DMD usually progresses rapidly in males. Patients with severe DMD may lose the ability to walk by age 12, and their respiratory system may stop functioning by approximately age 20 which usually results in death. In a less debilitating form of DMD, also known as Becker MD, dystrophin production is not shut down completely, but is reduced. For most DMD, the age of onset and rate of progression depends on how much dystrophin is produced and how well it functions in the cells.

There is currently no cure for muscular dystrophies, but medications and therapy may slow the progress of the disease. Respiratory therapy, physical therapy to prevent painful muscle contractures, orthopedic appliances used for support, and corrective orthopedic surgery may be needed to improve a patient's quality of life. Other treatments may include cardiac pacemakers and pharmaceuticals aimed at treating individual symptoms, for example corticosteroids can slow the rate of muscle deterioration, mild anesthetics can reduce pain, and antiepileptics can prevent seizures. Many of these treatments are ineffective and have severe side effects. There is therefore a need for a therapy that can prevent or slow the progress of muscular dystrophy with no or relatively milder side effects.

### SUMMARY OF THE INVENTION

The instrumentalities reported here provide a method for administering a pharmaceutical composition comprising an inhibitor of the nuclear factor kappa B (NFkappaB or NFκB) pathway in an amount that can inhibit or reduce the activation of NFκB in a subject diagnosed with muscular dystrophy. The present compositions and methods may be used to treat, prevent or reverse muscle damage or wasting caused by muscular dystrophy. More particularly, the disclosed compositions and methods are suitable for treating the form of muscular dystrophy caused by dystrophin deficiency.

In one aspect, this disclosure pertains to a method of administering a pharmaceutical composition. The methods may include diagnosing a subject that is in need of treatment for muscular dystrophy, administering to the subject an inhibitor of NFκB activation in an amount effective to inhibit nuclear activation of NFκB in said subject, and permitting the inhibitor to achieve therapeutic benefit for muscular dystrophy in the subject. By way of example, the NFκB inhibitor may include pyrrolidine dithiocarbamate, curcumin (diferuloylmethane), or their combinations.

NFκB plays an important role in the transcription activation of a large number of genes. For instance, many cytokines genes are activated by NFκB. It is shown here that the levels of some cytokines, such as IL-1β, IL-6 and TNFα, are elevated in the muscle of the mdx mouse model of muscular dystrophy. In another aspect of the present invention, chemicals or biological agents may be used to inhibit or reduce the production or secretion of these cytokines, and thus prevent or slow muscle degeneration in MD patients.

In another aspect of the present invention, the method of treatment may be enhanced by monitoring the effects of treatment, and adjusting treatment by increasing, reducing, or temporarily stopping treatment based on the result of monitoring. For instance, NFκB levels in the subject may be monitored to ascertain the status and effect of treatment. The total number of muscle fibers in skeletal muscles in the subject that are subjected to passive stretch during normal use may be monitored in order to ascertain the effect of treatment. In addition, the whole body strength of the subject may be measured during the course of the treatment.

Other parameters that may be monitored include the total tension generated by isolated muscles in the limbs of dystrophic subjects, the percentage of total cellular NFκB that is localized to the nuclear compartment of isolated dystrophic skeletal muscle, electrical properties and resting membrane potential of isolated dystrophic skeletal muscle fibers, the number of surviving striated muscle fibers in isolated skeletal muscles that are subjected to passive stretch during normal use, the total number of muscle fibers in skeletal muscles that are subjected to passive stretch during normal use, the number of skeletal muscle nuclei per muscle fiber in skeletal muscles that are subjected to passive stretch during normal use, the cross-sectional area of individual dystrophic muscle fibers in certain regions of skeletal muscle fibers that are subjected to passive stretch during normal use, the percentage of centrally located nuclei in muscle fibers that are subjected to passive stretch during normal use, and the total tension generated by isolated muscles in the limbs of dystrophic subjects.

In other aspects, the NFκB pathway is well documented in the art, and various inhibitors are available to regulate this pathway at one or more loci of pathway events. For example, an inhibitor may work by stabilizing the IκB protein and thereby preventing the NFκB from translocating into the nucleus. Another inhibitor may regulate the protein level of NFκB itself, yet other inhibitors may regulate the NFκB pathway by modulating the activity of nuclear NFκB.

As an alternative treatment method, a composition for use in the treatment of muscular dystrophy may contain a first inhibitor of NFκB activation in an amount that is effective to inhibit NFκB activation in the muscle cells of a subject, where the inhibitor of NFκB activation is effective to down-regulate the NFκB pathway at a predetermined first level. A second inhibitor of NFκB activation may then be used in an amount that is effective to inhibit NFκB activation in the muscle cells of a subject. The second inhibitor of NFκB activation is effective to down-regulate the NFκB pathway at a predetermined second level. Such predetermined second level is preferably different from the predetermined first level.

The two inhibitors may act on the same or different proteins in the NFκB pathway. In this manner, possible chronic side-effect of long term treatment may be mitigated by adjusting the ratio of the first and second inhibitors at intervals during a course of treatment. Adjustment may be on a regular periodic basis as specific cellular pathways regulating gene activation are modulated by the treatment and the particular drug combination becomes less efficacious, or as needed by assessment according to the aforementioned monitoring program.

In yet another embodiment, a subject may be treated with an inhibitor of NFκB activation in a first amount that is effective in bringing down the level of NFκB activation to a first level. After a period of treatment, a different amount of the same NFκB inhibitor is administered such that the level of NFκB activation is changed to a second level that is different from the first level achieved during the previous treatment period. In this manner, possible chronic side-effect of long term treatment may be mitigated by adjusting the level of NFκB inhibition. Adjustment may be on a regular periodic basis as specific cellular pathways regulating gene activation are modulated by the treatment and the particular drug combination becomes less efficacious, or as needed by assessment according to the aforementioned monitoring program.

### BRIEF DESCRIPTION OF THE DRAWINGS

FIG. 1 shows that acute in vivo PDTC administration increases cytosolic IκB-α levels in the mdx diaphragm (Western blot using anti-polyclonal IκB-α, # sc-371 antibody; Santa Cruz Biotechnology, Santa Cruz Calif.). Samples a and b were obtained using cytosolic extracts of diaphragm muscle in 2 untreated mdx mice following a single ip injection of saline at 3 (a) and 5 h (b) prior to sacrifice. Samples c and d were obtained from 2 littermates previously receiving a single 50 mg/kg ip dose of PDTC at 3 and 5 h prior to sacrifice, respectively. Densitometer measurements (Scion Image) yielded values (arbitrary linear units) of 198 (a), 336.8 (b), 1805.1 (c), and 1401.7 (d).

FIG. 2 compares the morphology of freshly isolated and fixed triangularis sterni (TS) muscles from age-matched control mdx (A, B) and PDTC-treated mdx mice (C, D). This muscle was chosen for study based upon the fact that it is chronically passively stretched and therefore exhibits profound dystrophic alterations and muscle fiber loss (Carlson et al., 2003). All photos are from the middle region of the TS at the same magnification (200×; calibration in A is 100 μm). (A) Untreated mouse TS at 9 months. (B) Saline-injected mouse (61 days) TS at 15 months. (C) PDTC-treated mouse TS at 9 months (30 days). (D) PDTC-treated mouse TS at 15 months (littermate to B, 56 days PDTC). TS muscle in panel A exhibited a few striated fibers (labeled “s”). The percent fibers in this area was 100% and the percent striated fibers was 22%. The small regions shown in brackets (C, D) lack fibers due either to hypercontraction or actual fiber loss. The 15-month TS region in panel B shows only a few fibers (labeled “i”) and no striated fibers. Arrow in panel B points towards one of two nerve branches present in this area. In contrast, the TS in panel D is from a 15-month PDTC-treated mouse and shows an area with many more striated fibers than in the saline-injected littermate (B).

FIG. 3 demonstrates that the methods used to assess the percent fibers and percent striated fibers in different regions of the mdx TS muscle provide an excellent determination of the loss of muscle fibers and the loss of striated muscle fibers in the dystrophic TS muscle. In this case, muscle fibers were examined in the cephalad, middle, and caudal regions of two non-dystrophic muscles that were fixed and examined by obtaining photographs of several microscopic areas within each of these regions as described in Carlson et al (2005). The results demonstrated that that the average percent fibers and percent striated fibers were approximately 100% in all regions of the non-dystrophic TS muscle.

FIG. 4 shows that daily treatment with PDTC (50-75 mg/kg ip; 27-30 days) increases the density of striated fibers in the TS of mdx mice aged 8.5-9 months at sacrifice (Series 1 experiments). The results were obtained from several sampled areas of intact and fixed TS muscles obtained from 3 PDTC-treated and 2 untreated littermate mdx mice (**P<0.01, t test). Shown are the N values for each condition (number of sampled areas, number of TS muscles). The areas sampled were in the middle region of the TS muscle. Black bars-untreated; gray bars—PDTC-treated mice.

FIG. 5 shows that daily treatment with PDTC (50 mg/kg ip; 48-76 days) increases the density of fibers in the TS of mature mdx mice aged 11.5-18.5 months (A, Series 2) and 21.5-22 months (B, Series 3) at sacrifice. N shows the number of areas and the number of mice sampled in each condition. Black bars: saline-injected mice; gray bars: PDTC-treated mice. A1 through A3 represent results obtained in caudal, middle, and cephalad thirds of the TS in Series 2, and B1 through B3 the corresponding results obtained in the older Series 3 mice. A4 and B4 represent corresponding summed results obtained over the entire TS (caudal, middle, cephalad regions). Symbols: XXX and εεε indicate a significant (P<0.001) effect of TS region on the percent fibers-for saline-injected and PDTC-injected mice, respectively (Kruskal-Wallis one-way ANOVA on ranks; Sigma Stat v 2.03). ααα indicates a significant effect of age (P<0.001) on the percent fibers for saline-injected mice in caudal regions and in the overall percent fibers (Mann-Whitney rank sum tests). *, **, and *** represent significant effects of PDTC treatment on percent fibers in comparison to corresponding saline-injected controls at three levels of significance (P<0.05, P<0.01, P<0.001, respectively; Mann-Whitney rank sum test).

FIG. 6 demonstrates that daily PDTC treatment increases the density of striated TS fibers in mdx mice aged 11.5-18.5 months (A, Series 2) and 21.5-22 months (B, Series 3) at sacrifice. The results were obtained from the same areas sampled for FIG. 4. Black bars: saline-injected mice. Gray bars: PDTC-injected mice. Symbols: X, ε, and εε indicate a significant effect of region on the percent striated fibers for saline-injected (X; P<0.05) and PDTC-treated mice (ε, εε; P<0.05 and P<0.01, respectively). αα indicates a significant effect of age (P<0.01) on the percent striated fibers in the caudal region, and a indicates a significant effect of age (P<0.05) on the overall percent of striated fibers for saline-injected mice (Mann-Whitney rank sum tests). *, **, and *** represent significant effects of PDTC treatment on the percent striated fibers in comparison to corresponding regions from saline-injected mice (P<0.05, P<0.01, P<0.001, respectively; Mann-Whitney rank sum tests).

FIG. 7 presents representative cross-sections (20 μm calibration) obtained from nondystrophic TS muscles (A) and from TS muscles from adult mdx mice treated chronically with vehicle (B) or PDTC (C). Staining is hematoxylin & eosin. (A) shows muscle fibers in the middle region from a 14.5 month nondystrophic mouse. (B) Dystrophic fibers in the caudal region of a 12.5 month old mdx mouse. Note the extensive fibrosis and cellular infiltration, and the centrally located nucleus in the middle of the section. (C) Caudal region of a 12 month old mdx mouse treated with PDTC for 48 days. Note the increase in number of fibers and myonuclei, the relative lack of centrally located nuclei, and the more densely stained pink cytoplasm in the PDTC treated preparation relative to the corresponding preparation from the vehicle-injected mouse (B).

FIG. 8 shows average histograms of fiber diameter for non-dystrophic TS muscles (A1-A3), TS muscles from mdx mice treated chronically with vehicle (B1-B3) and TS muscles from mdx mice treated chronically with PDTC (C1-C3). The entire TS muscle of each mouse was examined for the presence of muscle fibers and every section containing fibers was imaged at 95× magnification (e.g., FIG. 7) and sampled using Image J software. The histograms indicate the average number of events per preparation for each bin for each of the 3 regions of the TS muscle. The text indicates the number of animals for each condition and the total number of fibers per region. Note that all distributions have an approximately Gaussian profile with no evidence for distinct subpopulations of fibers. Note also the decrease in number of fibers that is evident in the caudal region of the mdx vehicle histogram (B1) and the increased number of fibers in the corresponding region of the PDTC treated preparations (C1).

FIG. 9 shows the average fiber diameters obtained for each of the 3 TS regions in nondystrophic, vehicle-injected mdx, and PDTC-injected mdx mice. Symbols: ννν, ωωω, and εεε indicate a significant (ANOVA, p<0.001) effect of region on fiber diameter for nondystrophic, mdx-vehicle, and mdx-PDTC treated preparations. *** indicates a significant reduction (t test, p<0.001) in fiber diameter in mdx preparations in comparison to corresponding nondystrophic regions. πππ indicates a significant difference between PDTC and vehicle-treated mice (t test, p<0.001). Note that PDTC apparently increased fiber number (FIGS. 8B1, C1) and reduced fiber diameter (FIG. 9) in the caudal regions, but significantly increased fiber diameter (FIG. 9) without altering fiber number (FIGS. 8B2,C2) in the middle TS region.

FIG. 10 shows the average fiber density for the 3 different regions of mature mdx TS muscles treated chronically either with vehicle or PDTC. The combined results from the Series 2 (average age 14.8 months at euthanasia; age matched vehicle and PDTC treated mice) and Series 3 (22 months at euthanasia; age matched vehicle and PDTC treated mice) experiments initially described in Carlson et al. (2005) were used to obtain the average fiber densities. This was accomplished by first sampling the entire TS muscle of each mouse for the presence of muscle fibers and then obtaining images at 95× for every section of each muscle that contained muscle fibers (e.g., FIG. 7). The images were obtained and further analyzed using Image J software. To obtain the average density of fibers for each region (caudal, middle and cephalad) of each TS muscle, a procedure was adopted to determine the length of each tissue cross section. Briefly, the width of each tissue section was determined at several points and divided by two to determine the midpoint of the tissue section at several points along the tissue section. These midpoints were then connected by individual lines using Image J and the total length through the middle of each section determined by summing the lengths of all the lines cutting through the middle of each section. The total number of fiber cross sections observed in each section was then divided by the total length through the middle of each section to obtain the fiber density for that particular section. The results were averaged for each region of each available TS muscle obtained from the Series 2 and 3 experiments initially reported in Carlson et al (2005). N refers to the number of TS muscles examined. Note the substantial decrease in the average density of fibers in the mdx vehicle treated preparations in comparison to the corresponding adult nondystrophic preparations. Note also that the PDTC treated preparations exhibited increases in the fiber density in the cephalad and caudal (σ, p<0.05) but not in the middle region, consistent with the histogram results shown in FIG. 8.

FIG. 11 shows that chronic treatment of an mdx mouse with PDTC for a period of 8.5 months decreases the loss of muscle fibers observed in the mdx TS muscle between 5 and 13.5 months. The average density of muscle fibers for each region of the TS muscle is shown for nondystrophic preparations and for vehicle injected mice at an average age of approximately 14.6 months. The density of fibers obtained from a 13.5 month mdx mouse treated chronically for 8.5 months with daily injections of PDTC (mdx-PDTC 8.5 months; 50 mg/kg) exhibited fiber densities that approached those seen in mature nondystrophic TS muscle and were much higher than the levels seen in mature vehicle-treated mdx mice.

FIG. 12 shows the average numbers of myonuclei per fiber cross section obtained from mature nondystrophic (ND) mice, mature mdx mice treated chronically with vehicle (mdx-veh) and mature mdx mice treated chronically with PDTC (mdx-PDTC). The combined results from the Series 2 (average age 14.8 months at euthanasia; age matched vehicle and PDTC treated mice) and Series 3 (22 months at euthanasia; age matched vehicle and PDTC treated mice) experiments initially described in Carlson et al. (2005) were used to obtain the average number of nuclei per sectioned muscle fiber. The entire TS muscle of each mouse was examined for the presence of muscle fibers and every section containing fibers was imaged at 95× magnification (e.g., FIG. 7) and sampled using Image J software. The total number of fiber cross sections and the total number of nuclei in each sectioned area of tissue were determined for every sectioned area of tissue that contained muscle fibers (e.g., FIG. 7). The number of nuclei per fiber was determined for each sectioned area by dividing the total number of nuclei in the area by the total number of fiber cross sections in the area. N refers to the number of sectioned areas and the number of TS muscles examined in each condition. There was a significant reduction (***, p<0.001; t tests) in the number of nuclei per fiber in both the mdx-vehicle and mdx-PDTC treated preparations in comparison to nondystrophic TS muscle. However, the TS muscles from the mature PDTC treated mice exhibited a significant (ε, p<0.05) increase in the number of nuclei per fiber in comparison to the vehicle-treated mice.

FIG. 13 shows that percent centronucleation is enhanced in mature mdx TS muscle fibers and significantly decreased by chronic treatment with PDTC. The combined results from the Series 2 (average age 14.8 months at euthanasia; age matched vehicle and PDTC treated mice) and Series 3 (22 months at euthanasia; age matched vehicle and PDTC treated mice) experiments initially described in Carlson et al. (2005) were used to obtain the percent centronucleation in vehicle-treated vs PDTC treated mature mdx mice. Percent centronucleation is defined as the percentage of myonuclei with a central as opposed to a peripheral location. Centrally located nuclei were operationally defined as nuclei situated at distances greater than 1 nuclear diameter away from the plasma membrane. In most instances, such nuclei were seen either in the approximate center of the muscle fiber or at approximately ¼ of the muscle fiber diameter away from the nearest plasma membrane. The entire TS muscle of each mouse used in the Series 2 and 3 experiments was examined for the presence of muscle fibers and every section containing fibers was imaged at 95× magnification (e.g., FIG. 7) and sampled using Image J software. Each nucleus observed in each sectioned area (e.g., FIG. 10) was scored as either central or peripheral and the percentage of central nuclei in each sectioned area was obtained by dividing the total number of centrally located nuclei in the area by the total number of myonuclei observed in the area and multiplying this value by 100. N refers to the number of sectioned areas and the number of TS muscles in each group. The small percent centronucleation seen in the nondystrophic TS is probably secondary to measurement error produced by a small number of tangential muscle sections. Both mature PDTC-treated and vehicle-treated TS muscles exhibited significant (*** −p<0.001) increases in percent centronucleation, but the PDTC treated mice exhibited significantly less (εε, p<0.01) centronucleation in comparison to the vehicle—injected group.

FIG. 14 shows that a 30 day treatment with 50 mg/kg PDTC beginning at 30 days of age significantly reduces the percent centronucleation observed in the mdx TS muscle at 60 days of age. N refers to the number of sectioned areas and the number of TS muscles in each group. PDTC treatment produced a significant (***, p<0.001) reduction in percent centronucleation in the PDTC treated mdx mice in comparison to the age matched mdx mice treated with 30 daily injections of vehicle.

FIG. 15 shows that Gd³⁺-sensitive resting Ca²⁺ currents are not responsible for a significant depression in resting potential observed in adult mdx TS muscle fibers. (A) Results from several non-dystrophic (C57BI10SnJ) TS preparations (N=number of impaled fibers, number of mouse muscles examined) obtained at ages of 5-17 months. Black histobar represents resting potentials in normal HEPES Ringer solution while lighter shaded bar indicates resting potentials from the same set of TS preparations obtained after adding 100 μM GdCl₃ to the solution surrounding the muscle fibers. (B) Corresponding results obtained from several mdx TS muscles (N=number of fibers, number of mdx mice) and indicates a significant (***P<0.001) decrease in resting potential in untreated adult mdx TS fibers (age 5-11.5 months) in comparison to the untreated nondystrophic fibers (A). The presence of 100 μM GdCl₃ did not significantly (P>0.05) alter the resting potential in the mdx TS muscle fibers (B) or in the nondystrophic fibers (A). The fibers impaled were in the caudal and middle regions of the TS muscle in all preparations.

FIG. 16 shows that daily administration of PDTC restores the resting potential to non-dystrophic levels in mice aged 8.5-9 months at sacrifice (Series 1 experiments). There was a significant (P<0.001) difference between the 3 treatment groups (one-way ANOVA) with Tukey test pairwise comparisons indicating significant differences in resting potential between the untreated nondystrophic (C57BI10SnJ) and mdx mice (***P<0.001) and between the PDTC-treated and untreated mdx mice (ααα, P<0.001). Shown are the means and standard errors and N refers to the number of TS fibers and muscle preparations (i.e., mice) in each group.

FIG. 17 shows that daily treatment with PDTC (50 mg/kg ip; 48-76 days) significantly increases the resting potential of fibers in the TS of mature mdx mice at 12.9 months (A, Series 2) and at 20 months (B, Series 3). N=number of impaled fibers, number of TS muscles. Shown are means and SE. (A1) Resting potentials from TS fibers of Series 2 (12.9 months average age at sacrifice) saline-injected mice are shown. Mean resting potentials were obtained in caudal and middle regions of the TS and throughout all regions of the TS (overall). (A2) Resting potentials from TS fibers of Series 2 PDTC-treated mice are shown. (B1) Resting potentials from TS fibers in Series 3 (average age 20 months) saline-injected mice. Shown are the mean resting potentials obtained in the caudal region and throughout the TS (overall). (B2) Resting potentials from TS fibers of Series 3 PDTC-treated mice are shown. Symbols: ααα represents a significant effect of age (P<0.001; Mann-Whitney rank sum test) on resting potential in the caudal region and in the overall sample of TS fibers in saline-injected mice. * and *** represent significant effects of PDTC treatment (P<0.05, P<0.001, respectively; Mann-Whitney rank sum tests) on resting potential in comparison to corresponding regions from saline-injected controls.

FIG. 18 presents measurements of forward pulling tensions (FPTs) produced by an mdx mouse using the noninvasive “whole body tension” measurement (Carlson and Makiejus, 1990). Individual increases in tension (upward deflections) represent individual pulling efforts in an attempt to escape into a darkened tube (stray marks indicate visually observed attempts).

FIG. 19 shows that the decline in forward pulling tension represented by the top 10 pulling attempts provides a measure of weakness in the mdx mouse that can be assessed noninvasively before and after a chronic period of drug administration. (A) shows results from a single saline-injected mouse on repeated “escape tests” performed prior to the daily administration of saline (Day 0) and after 9, 21, and 40 consecutive days of saline administration. (B) shows corresponding results from a PDTC injected mouse prior to (Day 0) and after several consecutive days (Day 9, 21,40) of PDTC administration (Series 2 experiments). For each individual “escape test” trial, the top 10 forward pulls were ranked from highest to lowest as indicated in FIG. 18. Each of the top 10 forward pulling tensions (measured in gms tension development/body weight in gms) were then divided by the highest tension developed during the trial (e.g., #1 in FIG. 19) and these results, normalized to the peak forward pulling tension, were then plotted against their rank order number for each trial. A linear regression was performed on the results to determine the proportionate decline in forward pulling tension over the top 10 pulls obtained for each trial. The negative slope of this decline is indicated next to each regression line (e.g. PDTC, Day 0: 0.0819) and is a measure of the average proportional decline in forward pulling tension per pull over the top 10 efforts. This value is termed the fatigue index (FI).

FIG. 20 demonstrates that chronic PDTC treatment reduces the fatigue index (FI) in the mdx mouse (Series 2 experiments). The FI is here defined as the negative slope of the decline in forward pulling tension/mouse body weight over the top 10 forward pulling tensions as described in FIG. 19 (e.g., 0.0819 at Day 0 for the PDTC treated mouse described in FIG. 19B). The effect of treatment was assessed by determining the proportional change in FI produced by either repeated saline injections (A-black bars) or PDTC injections (A-gray bars) at days 9, 21 and 40 for each of the two treatment groups. PDTC treated mice showed a significant effect of treatment on the relative fatigue (e, p<0.05; Krusal-Wallis ranks ANOVA; N=5 mice repetitively sampled) with significant differences observed for specific comparisons (ε, Tukey, p<0.05) between Day 0 and Day 9 and between Day 0 and Day 40. (B) shows the combined results over the course of treatment (Days 9, 21, 40) for the saline-injected and PDTC-injected mice (N=15 trials, 5 mice for each group) and indicates a significant (*, p<0.05; t test) effect of PDTC treatment in reducing FI in comparison to the saline-injected controls.

FIG. 21 shows that PDTC treatment produces functional improvement by significantly increasing whole body strength in mature mdx mice. Whole Body Tension measurements (WBT10, WBT5) are shown for age-matched mdx mice (range 5 months to 19.5 months at the beginning of the experimental period) treated chronically with PDTC as indicated in Carlson et al. ([2], Series 2 through 4). WBT measurements were obtained on each mouse (saline-injected or PDTC-injected) prior to treatment and on several occasions following initiation of the treatment. Shown are the mean and SE of the WBT 5 and WBT10 values (cf., Carlson and Makiejus) for either saline-injected (black bars) or PDTC-injected (gray bars) mice treated for at least 20 consecutive days. There was no significant effect of saline treatment on either measure (WBT 10 and WBT 5) when comparisons were made to pre-treatment values (not shown). However, PDTC treatment produced a significant (p<0.05) increase in WBT10 when comparisons were made to pre-treatment values (not shown). Post-treatment values indicate a significant (p<0.05, μ-t test, κ-Mann Whitney Rank Sum Test) effect of PDTC treatment on WBT10 and WBT5 in comparison to saline-treated controls. N refers to the total number of animals tested, the total number of WBT determinations.

FIG. 22 shows that daily treatment with PDTC prevents a decline in functional reserve (FR) normally seen in developing, young adult mdx mice. FR is defined as the average WBT10 value divided by the average WBT5 value for a WBT recording session (e.g., FIG. 18). Shown are the FR values obtained from an age matched sample of vehicle-injected mdx mice (black bars) and PDTC-treated mdx mice (gray bars). In each case, FR values were obtained prior to treatment at approximately 30 days of age and following 30 consecutive days of treatment at 60 days of age. The vehicle-injected mice exhibited an age-dependent significant decline in FR during this interval (αα, p<0.01) while the PDTC treated mice exhibited a slight increase in FR. The post-treatment FR of the PDTC treated mice was significantly higher than the corresponding value for the vehicle-injected mice (**). N indicates the number of mice in each treatment group (unfortunately, one of the vehicle-injected mice died during the experimental period).

FIG. 23 shows the Gastrocnemius Twitch amplitudes at Iₒ in nondystrophic (A) and mdx mice that were administered daily injections of saline (B) or PDTC (C). Horizontal calibration is 2 sec and vertical calibration is 10 gm. Note the difference in vertical calibration between the nondystrophic preparation (A) and the mdx preparations (B and C). (A) Untreated nondystrophic female mouse at 15 months of age, (B) MDX mouse at 15 months of age that had been treated with daily injections of saline vehicle (61 days treatment), (C) MDX mouse at 13.5 months of age that had been treated with daily injections of 50 mg/kg PDTC (61 days).

FIG. 24 shows that daily treatment with PDTC improves twitch tension development in mature mdx mice (average age 15 months at sacrifice). Results were obtained from Series 2 through 4 experiments (cf., Carlson et al., 2005). *** indicates significant difference (p<0.001, t test) between nondystrophic and vehicle-treated mdx preparations. PDTC treatment produced a 52% improvement (p=0.078) in twitch tension and a 45% improvement in twitch tension/muscle weight (p=0.058) that just failed to reach statistical significance (p>0.05). Additional experiments will be performed with mature mice (15 months) to determine whether PDTC has a significant effect on tension development at this age category.

FIG. 25 shows that daily treatment with PDTC improves twitch tension development in young adult mdx mice. MDX mice were treated for 30 days (beginning at 1 month of age) with either vehicle or PDTC (50 mg/kg) and measurements were obtained from the isolated gastrocnemius preparation at 2 months of age. Corresponding results were obtained from untreated 2 month old nondystrophic mice. N indicates the number of mice and corresponding number of isolated gastrocnemius preparations. * and ** indicate significant differences (p<0.05, p<0.01, respectively, t tests) between 2 month old vehicle-injected mdx mice and untreated nondystrophic mice. PDTC treatment induced a 13% improvement in twitch tension development and an 11% improvement in twitch tension/muscle weight (p>0.05) in comparison to vehicle-injected control mdx mice.

FIG. 26 uses trans AM assays of NFκB to show that chronic treatment with PDTC reduces total cellular NFκB (A) and increases the proportion of total cellular NFκB in the cytosolic fraction (B) in gastrocnemius muscle preparations. (C) shows the corresponding proportions in the nuclear fractions. Results were obtained from 2 vehicle-injected and 2 PDTC treated 2 month old mdx mice after a 30 day treatment period. In all cases, duplicate samples of 6 μg of protein from either cytosolic or nuclear fractions were used in the Trans AM assay after first determining that the total NFκB absorbance determined in the assay was linearly related to the sample protein concentration. Total NFκB absorbance was determined for each muscle as the sum of cytosolic and nuclear NFκB-specific absorbance for a total of 12 μg of protein (6 μg cytosolic protein, 6 μg nuclear protein). These results indicate that chronic PDTC treatment reduces the total cellular NFκB and the proportion of total cellular NFκB in the nuclear fraction. This assay is routinely used to screen compounds for their effects on NFκB localization in dystrophic skeletal muscle.

FIG. 27 shows that the cytosolic extracts of dystrophic (mdx) diaphragm exhibit elevated levels of NFκB dependent cytokines. The results (pg cytokine/μg protein) were obtained using standard protein determination (Lowry procedure) and ELISA techniques (Assay Designs, Inc.) on cytosolic extracts from the crural and costal regions of the diaphragm of nondystrophic (average age 18.4 months) and mdx (average age 14.6 months) mice. The values are expressed in pg of cytokine per mg total protein and were obtained from 5 nondystrophic and 5 mdx mice. There was a significant (*, p<0.05) increase in the levels of IL1-β in the costal diaphragm and significant (p<0.05) increases in IL6 in both the costal and crural diaphragms.

FIG. 28 shows that a single in vivo treatment with sulfasalazine (“SS”, Sigma Number S-0883; 100 mg/kg dissolved in HEPES Ringer solution and administered by intraperitoneal injection) may reduce nuclear NF-kappaB activation in the costal diaphragm of the mdx mouse. The nuclear extracts were prepared and analyzed by the electrophoretic mobility shift assay (EMSA: Gel Shift Assay System Promega Cat. # E3050 using NF-kappaB consensus oligonucleotide Promega Cat. # E3291 and gamma ³²P-ATP obtained from American Radiolabeled Chemicals Inc—Cat. # ARP-101). Lanes 1 (Gel 1 and Gel 2) are positive controls containing HeLa nuclear extract. Lanes 2 and 3 (duplicates in Gel 1 and Gel 2) are costal diaphragm nuclear extracts from 2 different mice treated 3 hours prior to euthanasia with a single vehicle injection. Lanes 4 and 5 (duplicates in gels 1 and 2) are corresponding nuclear extracts from 2 mdx mice treated 3 hours prior to euthanasia with a single injection of sulfasalazine. Lane 6 in Gel 1 is the nuclear extract from vehicle-injected sample (lane 2) incubated with excess unlabeled NFκB and lane 7 (Gel 1) is the same as lane 2 incubated with a nonspecific unlabeled oligonucleotide (AP-2). The primary band of NFκB binding is indicated by arrows and will be tested for specificity using the super-shift antibody technique.

FIG. 29 shows that a single in vivo treatment with parthenolide (PTN, Sigma Number P-0667; 5 mg/kg dissolved in HEPES Ringer solution containing 0.1% dimethylsulfoxide (DMSO) and administered by intraperitoneal injection) may reduce nuclear NFκB activation in the costal diaphragm of the mdx mouse. The nuclear extracts were prepared and analyzed by the electrophoretic mobility shift assay (EMSA: Gel Shift Assay System Promega Cat. # E3050 using NF-kappaB consensus oligonucleotide Promega Cat. # E3291 and gamma ³²P-ATP obtained from American Radiolabeled Chemicals Inc—Cat. # ARP-101). Gels 1 and 2 are duplicate gels. The lanes labeled with a “1” (Gel 1 and Gel 2) are positive controls containing HeLa nuclear extract. Lanes 2 and 3 (duplicates in Gel 1 and Gel 2) are costal diaphragm nuclear extracts from 2 different mice treated with a single vehicle injection 3 hours prior to euthasia. Lanes 4 and 5 (duplicates in gels 1 and 2) are corresponding nuclear extracts from 2 mdx mice treated with a single injection of pathenolide 3 hours prior to euthanasia. Lane 6 in Gel 1 is the nuclear extract from vehicle-injected sample (lane 2) incubated with excess unlabeled NFκB oligonucleotide and Lane 7 (Gel 1) is the same nuclear extract as lane 2 incubated with unlabeled nonspecific oligonucleotide (AP-2 consensus sequence).

FIG. 30 shows that TNFα expression in costal diaphragm is reduced following a single injection of sulfasalazine (SS; 100 mg/kg, ip) administered 3 hours prior to euthanasia. The results were obtained from the cytosolic extracts of 5 vehicle injected mdx mice and SS treated mice at 3 to 5.5 months of age and are expressed as pg of cytokine per mg total protein.

FIG. 31 shows that the expression of IL1-β in cytosolic extracts of mdx muscle depends upon the muscle origin and is not influenced by a single injection of sulfasalazine (SS; 100 mg/kg, ip) administered 3 hours prior to euthanasia. The results were obtained from the same cytosolic extracts used in FIG. 30 and indicated that the expression of IL1-β is significantly (αα and ααα, p<0.01 and p<0.001; ANOVA followed by Tukey pairwise comparisons) increased in the costal and crural diaphragm in comparison to the mdx gastrocnemius muscle.

FIG. 32 shows that the expression of IL6 in cytosolic extracts of mdx muscle depends upon the muscle origin and is reduced by a single injection of sulfasalazine (SS; 100 mg/kg, ip) administered 3 hours prior to euthanasia. The results were obtained from the same cytosolic extracts used in FIG. 30 and indicated that the expression of IL6 is significantly (αα, p<0.01; ANOVA followed by Tukey pairwise comparisons) increased in the costal and crural diaphragm in comparison to the mdx gastrocnemius muscle. MDX mice treated with a single injection of SS exhibited reduced levels of IL6 in the cytosolic extracts of both the costal and crural diaphragms.

FIG. 33 shows that daily treatment with sulfasalazine (SS; 100 mg/kg; intraperitoneal, 68 days) significantly improves the resting membrane potential in the TS muscle. Average resting potential obtained from the TS of a mouse treated chronically with 70 daily injections of vehicle (HEPES Ringer—in mM: 147.5 NaCl, 5 KCl, 2CaCl2, 11 glucose, 5 HEPES; black histobar) are compared to the average resting potential obtained from the TS of a littermate mouse treated with 68 daily injections of SS (gray histobar). The mice were 7 months of age at the time the recordings were made. Shown are the means plus SEM. N refers to the number of fibers, number of TS muscles. ** indicates that the resting potential of the sample of SS—treated fibers (N=21) was significantly (p<0.01) larger than that of the sample of vehicle—treated fibers.

### DETAILED DESCRIPTION

The design of a more effective treatment for Duchenne and related dystrophinopathies depends upon an improved understanding of the pathogenesis of these disorders. Since the initial discovery of dystrophin, considerable attention has been placed on understanding those mechanisms by which the absence of this cytoskeletal protein in Duchenne muscular dystrophy and in the dystrophic (mdx) mouse (Bulfield et al., 1984, Hoffman et al., 1987 and Koenig and Kunkel, 1990) ultimately leads to muscle necrosis. A primary hypothesis based upon the membrane localization of dystrophin (Arahata et al., 1988) and the subsequent characterization of the transmembrane dystrophin-glycoprotein complex (Ervasti and Campbell, 1991) suggests that dystrophin forms a structural bridge that supports the plasmalemma by physically interacting with extracellular components of the basal lamina (Matsumura and Campbell, 1994). An alternative hypothesis suggests that alterations in ion channel function and associated local increases in Ca²⁺ influx may occur when ion channels aggregate in association with a dystrophic cytoskeleton (Carlson, 1998). Each of these hypotheses generally involve secondary increases in Ca²⁺ influx that occur as a result of the structural breakdown of the plasma membrane or by the formation of abnormal ion channel-cytoskeletal interactions at specific ion channel aggregates (Carlson, 1998).

Muscular Dystrophy (MD) is neuro-muscular disease with a diverse range of manifestation and pathogenesis. The diagnosis of MD may thus utilize a wide range of clinical tools. Although behavioral diagnosis may be the primary tool to spot the disease, neurological, histological, biochemical, or genetic testings may be used to more definitively diagnose the disease (See generally, El-Bohy and Wong, 2005). Physical methods such as microwave or NMR imaging may also aid a clinician in the diagnosis of MD. The compositions and methods disclosed herein may be used to treat or to slow the progress of a patient who has been diagnosed with Muscular Dystrophy. These compositions may also be useful for a subject who may not have the typical symptoms of MD but who is otherwise in need of such a therapy. By way of example, the compositions and methods may prove useful for a subject who may be genetically predisposed to MD based on family history but who may not have yet developed any symptoms of MD. Such person may be treated in a prophylactic way to prevent or delay the onset of symptomatic disease.

Although fluorometric and electrophysiological investigations provided evidence for enhanced resting Ca²⁺ influx in cultured dystrophic (mdx) myotubes (Carlson et al., 2001 and Tutdibi et al., 1999), Mn²⁺ quench determinations did not indicate differences in resting Ca²⁺ influx between nondystrophic and mdx dissociated adult flexor digitorum brevis muscle fibers (DeBacker et al., 2002). Results obtained in this laboratory also indicated that extrajunctional resting Ca²⁺ influx was not elevated in severely dystrophic and intact undissociated adult mdx skeletal muscle fibers (Carlson et al., 2003), and demonstrated that extrajunctional increases in resting Ca²⁺ influx are therefore not pathogenic in muscular dystrophy. These investigations also showed that the mdx triangularis sterni (TS) muscle exhibits severe dystrophic alterations with fibrosis, fat infiltration, hypercontraction, dissolution of myofibrillar material, cytoplasmic rarefaction with delta lesions, and substantial decreases in the number of striated muscle fibers and in the total number of muscle fibers. This loss of muscle fibers continues until the mdx TS becomes a thin layer of connective tissue with only a few remaining muscle fibers by about 1.5-2 years of age (Carlson et al., 2003).

Functional studies indicate that the TS is an expiratory muscle that is chronically passively stretched to about 107% of its resting length and concentrically activated at a rate of approximately 250 times per minute (DeTroyer and Ninane, 1986, Hwang et al., 1989, Gosselin et al., 2003 and Ninane et al., 1989). The severe dystrophy seen in this mdx muscle therefore strongly suggests that physical factors or signaling pathways activated by passive stretch play central roles in the pathogenesis of dystrophic muscle (Carlson et al., 2003). Such factors and/or pathways would presumably also be involved in the susceptibility of dystrophic fibers to the damaging effects of eccentric muscle contractions (Petrof et al., 1993 and Weller et al., 1990).

A potential pathway that may be involved in stretch-dependent dystrophic pathogenesis (Carlson et al., 2003) is suggested from results described by Kumar and Boriek (2003), who showed that a single 15-min period of passive stretch increased the nuclear activation of the transcription factor NFκB by two times in isolated nondystrophic muscle fibers, and that resting mdx muscle fibers from 15-day-old mice exhibited nuclear NFκB levels that were approximately two times those seen in age-matched nondystrophic fibers. The stretch-dependent increase in nuclear activation of NFκB in nondystrophic muscle did not require Ca²⁺ influx and was associated with a reduction in cytosolic levels of IκB-α secondary to the activation of IκB kinase (IKK; Kumar and Boriek, 2003). These results are consistent with stretch-dependent activation of the classical NFκB pathway in which IKK phosphorylates IkB-α, thus tagging it for future ubiquitination and proteasomal degradation (Karin et al., 2004). This process disassociates IkB-α from the dimeric p50/p65 NFκB, thus allowing the dimer to enter the nucleus and activate an array of NFkappaB-dependent genes. These genes include several proinflammatory cytokines (e.g., IL-1B, II-2, IL-6, IL-8, TNF-α), chemokines (IL-8, RANTES), inducible enzymes (iNOS, cyclooxygenase), and adhesion molecules (ICAM, VCAM; Barnes, 1997, Li et al., 2002 and Siebenlist et al., 1994). Because some of the genes activated by NFκB encode proteins that are beneficial to the muscles and promote cell division and cell survival (e.g. cyclin D1, bcl-2, bclₓₗ, cellular inhibitor of apoptosis 1 (clAP1), clAP2, xIAP), while other genes encode proteins that are pro-inflammatory, the elevated NFκB activity may be either compensatory or detrimental to the structure and function of dystrophic skeletal muscle. The effects of NFκB activation in dystrophic muscle may be determined by chronically inhibiting the NFκB pathway and determining the effects of this inhibition on the structure and function of dystrophic skeletal muscle.

NFκB is a transcription factor that plays an important role in many cellular processes. In its inactive state, NFκB resides in the cytoplasm and is bound to another protein called IκB. Upon cell activation, IκB may be modified and targeted for degradation. The freed NFκB may then translocate into the nucleus, and along with other transcription factors, activate transcription of target genes. (See Karin et al., 2004, for a general discussion of the NFκB pathway).

NFκB activation refers to a state of the NFκB molecule that is capable of participating in transcription activation. Inhibitors of NFκB activation generally refer to an agent that either partially or completely blocks NFκB participation in the activation of many its target genes. A large number of NFκB target genes have been reported in the literature. The mRNAs of these target genes are normally present at low levels and their levels increase dramatically when NFκB and other transcription factors bind to regulatory elements of these genes and activate their transcription.

To evaluate the effect of various chemical agents on animal models of MD, experimental male and female mdx mice may be injected intraperitoneally (ip) with various chemicals at different dosage. In the case of PDTC, 50-75 mg/kg of PDTC (obtained from Sigma) may be dissolved in a saline solution (HEPES-Ringer; in mM: 147.5 NaCl, 5 KCl, 2CaCl₂, 11 glucose, 5 HEPES, pH 7.35) for injection. More particularly, mdx mice aged 5-22 months at the beginning of the treatment may receive daily injections of PDTC at doses between 50 and 75 mg/kg for a period of 27-30 consecutive days. The mice may be sacrificed after 1-24 months from the beginning of treatment to determine the effect of the treatment with the chemicals.

In one embodiment, the treatment effect may be evaluated by comparing the morphological results between 3 regions of the triangularis sterni (TS); the caudal third of the muscle extending toward the xiphoid process, the middle third, and the cephalad third of the muscle. This procedure is adopted because there are obvious differences between these regions in the extent of pathology and the muscle thickness in the mdx TS (not clearly seen in the nondystrophic TS) even when examining the muscle at low power (e.g., 4×). Results from animals that die during the experimental time frame (both saline injected and PDTC treated) are typically excluded from the study. Although the current sample of PDTC-treated mice is not sufficient to determine any toxic effects of the drug, no obvious effect of PDTC treatment on viability is observed in the overall sample of mice used in these studies.

Conventional intracellular recording techniques may be used to determine resting membrane potential in individual TS muscle fibers from nondystrophic (C57BI 10SnJ) and mdx (C57BI10-mdx) mice (For detailed description, see Carlson and Roshek, 2001). After removing the TS muscle preparation (See Carlson et al., 2003), the entire TS muscle may be placed in a specialized recording chamber and stretched across a thin glass coverslip using specialized dissecting hooks (manufactured locally) that are attached to the sternum and to small (1-2 mm) cut sections of the ribs. The preparation may be placed in the chamber with the external surface of the TS facing upwards and is minimally stretched to its approximate length in situ (about 95-105% of resting length).

The chamber is typically filled with a small volume (2 ml) of normal HEPES Ringer solution at room temperature and fiber-filled glass micropipettes (3 M KCl; R=20-70 MΩ) may be used to impale individual fibers at an angle of 90° to the principal fiber axis. Signals may be amplified with a Warner Instruments Model IE 201 electrometer and displayed on an oscilloscope. Individual fibers may be viewed using an Olympus IMT2F microscope equipped with long working distance (20×, 40×) objectives. Impalements may be obtained after first electrically balancing the recording system (0 mV output relative to ground) and viewing the electrode tip over a muscle fiber. The electrode may be slowly advanced and inserted into the muscle fiber by gently tapping the manipulator or temporarily unbalancing the negative capacitance of the recording circuit. The voltage deflection associated with membrane insertion is noted, and each recording may be maintained for a few minutes before the electrode is rapidly withdrawn from the fiber. The voltage deflection associated with withdrawal from the cell is also noted and the larger of the two deflections (i.e., insertion or withdrawal) is usually taken as the fiber resting potential. Differences between the insertion and withdrawal voltage deflections are generally 0-4 mV.

Resting potentials from approximately 20 fibers may be obtained from each isolated TS muscle over a total recording period of about 1.5 h. The presence or absence of miniature endplate potentials is also noted to identify endplate from nonendplate regions. When no attempt is made to identify endplates, approximately 97% of the recordings may be from nonendplate regions.

Morphological assessment of total fiber density and density of striated fibers may be conducted as described in the following text. Immediately after completing the resting potential measurements, the minimally stretched TS muscle preparations attached to the dissecting hooks may be fixed overnight in 2% glutaraldehyde (0.1 M cacodylate buffer) and subsequently washed several times in 0.1 M cacodylate. Before removing the preparation from the recording chamber, microphotographs of approximately 24 randomly sampled areas may be obtained at 200-300× magnification in caudal, middle, and cephalad regions of the TS. In the initial studies, a smaller number of photographs may be obtained over the middle portion of the TS muscle.

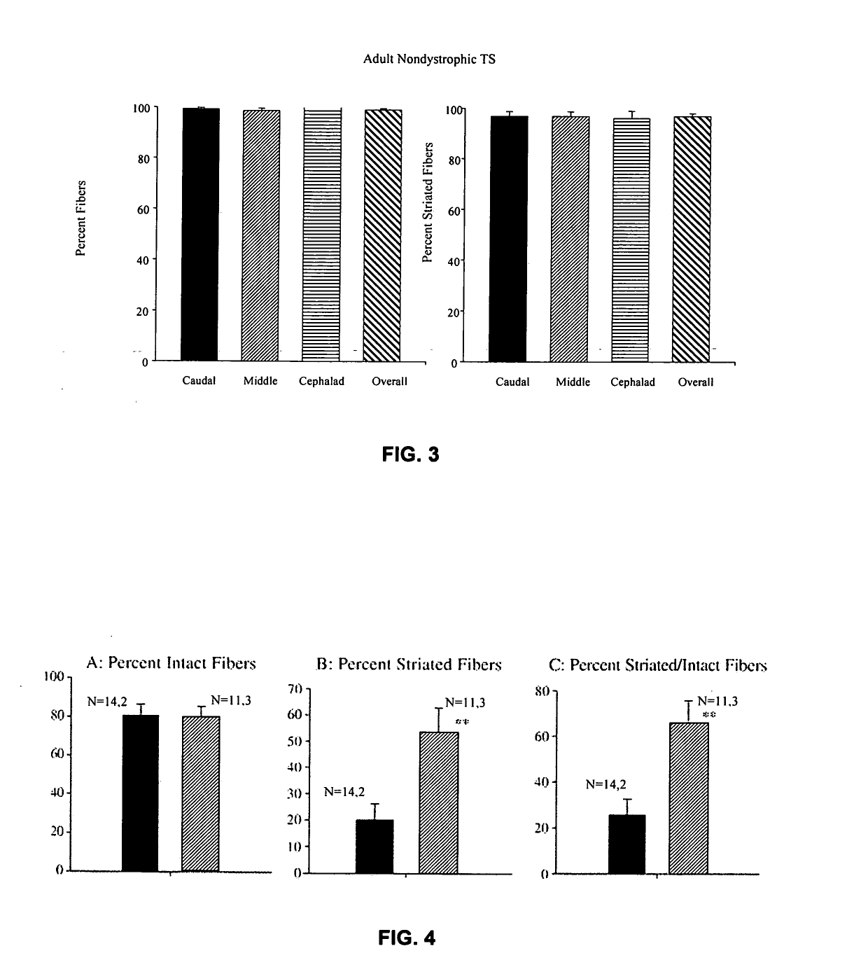

Each preparation may be illuminated using bright-field optics to minimize depth of focus issues in visualizing muscle fiber striations. Since the mdx TS muscle is a flat and thin preparation, all the fibers in each area are roughly within the same focal plane. However, small variations in depth of focus may produce small variations in the appearance of striated fibers. Such effects may be minimized by routinely adjusting the plane of focus to maximize the number of striated fibers in each photographed area. The density of muscle fibers and the density of striated fibers may then be evaluated for each sampled area by drawing a line orthogonal to the principal axis of the TS muscle fibers across the entire viewing area of each photograph. The percentage of the length of this line that covered muscle fibers and the percentage of this line that covered striated muscle fibers (defined as having striations over at least 50% of the observed length) may be used to determine the percentage of muscle fibers and striated muscle fibers, respectively, for each photographed muscle area. Control experiments using these procedures on adult nondystrophic TS muscles (N=37 areas, 2 TS muscles at 19 and 27 months) may yield average values of 99.1±0.5 (SE) percent fibers and 96.7±1.2 percent striated fibers with no regional differences across the caudal, middle, and cephalad thirds of the muscle (FIG. 3).

Cytosolic levels of IκB-α may be determined using Western blot techniques. Cytosolic and nuclear fractions may be obtained from isolated diaphragm muscles using the techniques described in Kumar and Boriek, 2003. Briefly, the muscles may be weighed after removing tendinous components, and frozen and homogenized by mortar and pestle in lysis buffer on ice (1 mg muscle/18 μl lysis buffer containing 10 mM HEPES, 10 mM KCl, 1.5 mM MgCl₂, 0.1 mM EDTA, 0.1 mM EGTA, 1 mM dithiothreitol, 0.5 mM phenylmethylsulfonylfluoride, 2.0 μg/ml leupeptin, 2.0 μg/ml aprotinin, 0.5 mg/ml benzamidine, at pH 7.9). To lyse the cells, the ground tissue may be subjected to two freeze-thaw cycles and subsequently vortexed and centrifuged (13,000 rpm, 10 s). The supernatant cytosolic extract may be immediately frozen (−80° C.) for Western blot analyses, while the nuclear pellet may be resuspended on ice in a nuclear extraction buffer (20 mM HEPES, 420 mM NaCl, 1 mM EDTA, 1 mM EGTA, 25% (v/v) glycerol, 1 mM dithiothreitol, 0.5 mM phenylmethylsulfonylfluoride, 2.0 μg/ml leupeptin, 2.0 μg/ml aprotinin, 0.5 mg/ml benzamidine; at pH 7.9) at a ratio of 4 μl per milligram of muscle weight. The preparation may be incubated on ice with intermittent vortexing before being centrifuged as described above for 5 min. at 4° C. The supernatant nuclear extract may then be frozen (−80° C.) for subsequent biochemical determinations.

Equal amounts of proteins (based on Lowry Assay) from treated vs. untreated cytosolic fractions may be boiled in SDS-PAGE sample buffer for 5 min, applied to 10% SDS-PAGE gel, and blotted onto PVDF (polyvinyl difluoride) membrane. The membrane may be blocked with 5% milk in TBST and immunoprobed with anti-polyclonal IκB-α, # sc-371 (Santa Cruz Biotechnology, Santa Cruz, Calif.), at 1:500 dilution in 5% milk-TBST overnight at 4° C. After three washes, the membrane may be incubated with a 1:10,000 dilution of the appropriate peroxidase-conjugated secondary antibody for 1 h at room temperature. After additional washing steps, the antibody complex may be detected by chemiluminescence using the ECL detection reagent (Amersham) and densitometric measurements may also be obtained. Protein loading levels may be examined by staining the membrane with Coomassie blue dye.

The compositions and methods disclosed here provide a therapy for MD by administering a chemical or biological agent to a subject to modulate the nuclear factor kappa B (NFkappaB or NFκB) pathway such that the activation of NFκB is either inhibited or reduced in the subject's muscle tissues. The phrases “inhibitor of activation” and “NFκB inhibitor” are used interchangeably to refer to an agent that decreases or reduces the transcriptional or other activities attributable to NFκB in the cells.

In one embodiment, an NFκB inhibitor may be an agent that reduces the production of NFκB protein in the cells. In another embodiment, an NFκB inhibitor may be an agent that stabilizes the IκB protein. In another embodiment, an NFκB inhibitor may be an agent that blocks the translocation of NFκB into the nucleus. In yet another embodiment, an NFκB inhibitor may be an agent that prevents NFκB from acting as a transcription factor after its translocation into the nucleus.

For purpose of the present disclosure, the term “subject” refers to any animal, including for example, mice, rats, dogs, guinea pigs, rabbits and primates. In the preferred embodiment, the subject is human. While the methods disclosed herein may be used most often in humans, they may also be applied to other animals. The terms “treating” (or “treatment”) means slowing, stopping or reversing the progression of a disorder. In the preferred embodiment, it means reversing the disorder's progression, ideally to a point of elimination. The term “chronic” means any period of time that lasts over 30 days.

Agents for NFκB inhibition may be chemicals, either inorganic or organic, that show an inhibitory effect on NFκB activation, and combinations thereof. Agents may also include extracts obtained from natural sources, such as those from plants, animals, worms, lower eukaryotes such as fungi, or microorganisms. Agents may also be selected from oligonucleotides, proteins, peptides, or compositions containing antibodies, and combinations thereof.

The term “cytokine” refers to proteins released by cells that have a specific effect on the interactions between cells, on communications between cells or on the behavior of cells. Cytokines include interleukins, lymphokines and other cell signaling molecules, such as tumor necrosis factor and the interferons. “NFκB dependent cytokines” means those cytokines whose gene transcription requires activation of NFκB. “Immunoassay” means any assays that utilize an antibody or an antiserum.

The terms “protein,” “polypeptide,” and “peptides” are used interchangeably in this disclosure. A pharmaceutical composition is a mixture containing more than one chemical, or more than one protein. “Inhibit” or “inhibition” means lessening, reducing, attenuating a cellular activity. “Inhibitor” means any agent that is capable of inhibition. The term “substantially” means more than 40%. For example, when the level of a protein is substantially reduced, its level decreases by 40% or more.

Examples of NFκB inhibitors may include carbamates, such as pyrrolidine dithiocarbamate (PDTC), curcumin (diferuloylmethane), and combinations thereof. Other compositions that function to block NFκB activation are also useful. All the references cited in this paper are incorporated by reference to the same extent as though fully replicated herein. By way of examples, these compositions include, but are not limited to:
(1) SN-50, a cell-permeable peptide that inhibits the nuclear translocation of NFκB (D'Acquisto et al., Naunym-Schmiedeberg's Arch. Pharmacol. 364, 422, 2001);
(2) Gabaexate mesilate, a serine protease inhibitor which stabilizes cytosolic IκB-α levels in the presence of tumor necrosis factor (TNF) and decreases the nuclear activation of NFκB (Crit. Care Med., 31(4), 1147, 2001; Yuksel et. al., J. Pharmacol. Exp. ther., 305(1), 2003);
(3) BMS-345541, which stabilizes cytosolic levels of IκB-α by inhibiting IκB kinase (IKK, Burke et al., J. Biol. Chem., 278 (3), 1450-1456, 2003);
(4) The following compounds specifically identified in a review article summarizing several additional agents which inhibit IKK and thereby stabilize or otherwise increase cytosolic levels of IκB-α (Karin et al., 2004—see Table 1):
   (a) The “quinazoline analogue” identified as Compound 1 or SPC-839,
   (b) The “Beta-carbolin analogue” identified as Compound 2 or PS-1145,
   (c) The “amino-thiophenecarboxamide derivative” identified as Compound 4 or SC-514,
   (d) The “ureido-thiophenecarboxamide derivative” identified as Compound 5,
   (e) The “diarylpyridine derivative” identified as Compound 6,
   (f) The “anilino-pyrimidine derivative” identified as Compound 7,
   (g) The “pyridooxazinone derivative” identified as Compound 8,
   (h) The “indolecarboxamide derivative” identified as Compound 9,
   (i) The “benzoimidazole carboxamide derivative” identified as Compound 10,
   (j) The “pyrazolo(4,3-c) quinoline derivative” identified as Compound 11,
   (k) The “imidazolylquinoline-carboxaldehyde semicarbazide derivative” identified as Compound 12,
   (l) The “amino-imidazolecarboxamide derivative” identified as Compound 13, and
   (m) The “pyridyl cyanoguanidine derivative” identified as Compound 14;
(5) Epigallocatechin-3-gallate and similar polyphenols extracted from Green tea which inhibit the nuclear activation of NFκB by stabilizing cytosolic levels of cytosolic IκB-α (Singh et al., Arthritis and Rheumatism, 46 (8), 2079, 2002);
(6) Diethyidithiocarbamate which inhibits the nuclear activation of NFκB (Xuan Y.-T. et al., Circulation Research, 84, 1095-1109, 1999; Blondeau et al., The J. of Neuroscience, 21(13), 4668, 2001);
(7) The κB decoy DNA sequence identified by Blondeau et al (The J. of Neuroscience, 21(13), 4668, 2001) which inhibits the nuclear activation of NFκB;
(8) The proteasome inhibitor MG 132 which inhibits the degradation of cytosolic IκB-α and reduces the nuclear activation of NFκB (Takeuchi et al., Digestive Diseases and Sciences, 47(9), 2070, 2002);
(9) The agent diferuloylmethane (curcumin) which is present in yellow curry and which reduces the nuclear activation of NFκB by stabilizing cytosolic IκB-α (Singh and Aggarwal, J. Biol. Chem., 270 (42), 24995, 1995);
(10) The cell-permeable synthetic peptide (NBD peptide; Leu-Asp-Trp-Ser-Trp-Leu) identified by May et al. (Science, 289, 1550, 2000) which inhibits IKK activity and reduces the nuclear activation of NFκB by interfering with specific binding reactions of the IKK complex that are required for IKK activity;
(11) The herbicide 3,4-dichloropropionaniline (propanil) which inhibits nuclear NFκB activation in response to bacterial lipopolysacharides in macrophages (Frost et al., Toxicol. and Applied Pharmacology, 172, 186, 2001);
(12) The water soluble extract of Uncaria tomentosa (cats claw) termed C-Med 100 which inhibits the nuclear activation of NFκB without influencing the stability of cytosolic IκB-α (Akesson et al., Internat. Immunopharmacol., 3, 1889, 2003);
(13) The aqueous extract of Uncaria tomentosa (obtained by boiling cats claw bark for 30 minutes) identified by Sandoval-Chacon et al. (Aliment. Pharmacol. Ther., 12, 1279, 1998) that inhibits the nuclear activation of NFκB;
(14) The hydro-alcoholic extract of Uncaria tomentosa identified by Aguilar et al (J. of Ethnopharmacol., 81, 271, 2002) which inhibits the nuclear activation of NFκB;
(15) Dehydroxymethylepoxyquinomicin (DHMEQ) which inhibits the nuclear translocation of NFκB in cultured Jurkat cells (Ariga et al, J. Biol. Chem., 277 (27), 24625-24630, 2002) and inhibits the nuclear activation of NFκB in obstructed kidneys (Miyajima et al., J. of Urology, 169, 1559-1563, 2003);
(16) Pirfenidone(2(1H)-Pyridinone, 5-methyl-1-phenyl) which inhibits nuclear NFκB activation in cultured hepatocytes exposed to the cytokine IL-1β (Nakanishi et al., J. of Hepatology 41, 730-736, 2004);
(17) The agents, Bay 11-7085 and Bay 11-7082, which inhibit the nuclear activation of NFκB by inhibiting IKK-dependent phosphorylation of the inhibitory protein IκB-α in a variety of cell types (Izban et al., Hum. Pathol., 31(12), 1482-1490, 2000; Mabuchi et al., Clin. Cancer res., 10(22), 7645-7654, 2004; Zou et al., Am. J. Physiol. (Gastrointest Liver Physiol), 284(4), G713-G721, 2003);
(18) Gliotoxin, which decreases the nuclear activation of NFκB by increasing the concentration of cytosolic phosphorylated IκB-α (Pahl et al., J. Exp. Med., 183, 1829-1840, 1996);
(19) The class of sesquiterpene lactones derived from a variety of botanical sources of the Asteracae family including Artemisia annua (Aldieri et al., FEBS Lett, 552, 141-144, 2003), Achillea millefolium (Mustakerova et al., Verlag der Zeitschrift fur Naturforschung, 57c, 568-570), Amica montana and Amica chamissonis (Lyβ et al., J. Biol. Chem. 273 (50), 33508-33516), Tanacetum parthenium (“feverfew”; Jan and Kulkarni, J. Ethnopharmacol, 68, 251-259, 1999), Mikania guaco, Milleria quinqueflora, Vanillomopsis arborae, Proteopsis furnensis, Eremanthus mattogrossensis, Tithonia diversifolica (Rungeler et al., Biorganic and Medicinal Chemistry, 7, 2343-2352, 1999), and several species of the genus Carpesium (C. macrocephalum, C. lipskyi, C. Cemuum, C. longfolium; Shi et al., Planta Med., 65, 94-96, 1999; Yang et I., Pharmaxie 56, 825-827, 2001; Yang et al., Planta Med., 68, 626-630, 2002; Yang et al., J. Nat. Prod., 66, 1554-1557, 2003) to include the following specific compounds:
   a.) Parthenolide, which inhibits the nuclear activation of NFκB in Jurkat T leukemia cells, HeLa cells, mouse L929 fibroblasts, and rat aortic smooth muscle cells by inhibiting the degradation of cytosolic IκB-α (i.e., stabilizing cytosolic IκB-α) in the presence of a variety of agents that stimulate the NFκB pathway (Hehner et al., J. Biol. Chem., 273(3), 1288-1297, 1998; Wong and Menendez, Biochem. Biophysica Res. Comm., 262, 375-380, 1999). It has also been shown to inhibit IKK activity and the nuclear activation of NFκB in HeLa cells (Kwok et al., Chem and Biol., 8, 759-766, 2001) and to inhibit the binding of NFκB to the κB consensus sequence in nuclear extracts of rat lungs (Sheehan et al., Molec. Pharmacol., 61, 953-963, 2002).
   b.) Artemisinin, which blocks the nuclear activation of NFκB in cultured human astrocytoma T67 cells exposed to a mix of lipopolysacharrides and cytokines (Aldieri et al., FEBS Letters, 552, 141-144, 2003).
   c.) Helenalin, which inhibits nuclear activation of NFκB by TNFα in Jurkat T cells by alkylating the p65 subunit and inhibiting binding to the κB consensus sequence (Lyβ et al., J. Biol. Chem., 273, 33508-33516, 1998; cf Rungeler et al., Biorganic and Medicinal Chemistry, 7, 2343-2352, 1999).
   d.) Mexicanin I, which inhibits the nuclear activation of NFκB in TNFα stimulated Jurkat T cells (Lyβ et al., J. Biol. Chem., 273, 33508-33516, 1998).
   e.) 2,3-Dihydroaromaticin, which inhibits the nuclear activation of NFκB in TNFα stimulated Jurkat T cells (Lyβ et al., J. Biol. Chem., 273, 33508-33516, 1998).
   f.) Helenalin-isobutyrate, which inhibits the nuclear activation of NFκB in TNFα stimulated Jurkat T cells (Lyβ et al., J. Biol. Chem., 273, 33508-33516, 1998).
   g.) Isohelenin, which stabilizes cytosolic IκB-α and inhibits the nuclear activation of NFκB in cultured rat aortic smooth muscle cells (Wong and Menendez, Biochem. Biophysica Res. Comm., 262, 375-380, 1999).
(20) Arctigenin and related dibenzylbutyrolactone lignans such as demethyltraxillagenin, which inhibit the nuclear localization of NFκB by stabilizing cytosolic IκB-α in Raw 264.7 mouse macrophages (Cho et al., International Immunopharmacology, 2, 105-116, 2002);
(21) Sulfasalazine, which inhibits the nuclear activation of NFκB in cultured human colonic epithelial cells (Wahl et al., J. Clin. Invest., 101, 1163-1174, 1998), in human colonic biopsies from individuals treated with chronic oral doses of the drug (Gan et al., J. of Gastroenterology and Hepatology, 20, 1016-1024, 2005), in human adipose tissue and skeletal muscle explants from biopsies obtained from healthy pregnant women (Lappas et al., Endocrinology, 146 (3), 1491-1497, 2005), in human glioblastomas (Robe et al., Clinical Cancer Research, 10, 5595-5603, 2004), and in Jurkat T cells (Weber et al., Gastroenterology, 119, 1209-1218, 2000) by a mechanism involving stabilization of cytosolic IκB-α (Wahl et al., 1998) and inhibition of IKK activity (Weber et al., 2000);
(22) Guggelsterone, which inhibits the nuclear activation of NFκB by inhibiting IKK in a variety of cancer cells in which enhanced nuclear NFκB is either constitutive or secondary to external activation of the NFκB pathway by a variety of agents (Shishodia and Aggarwal, J. Biol. Chem., 279 (45, November 5), 47148-47158, 2004);
(23) Troglitazone, which inhibits the nuclear activation of NFκB in mononuclear leucocytes by reducing total NFκB levels and increasing total IκB-α levels in obese humans treated with daily oral doses of the drug (Ghanim et al., J. of Clin. Endocrinology and Metabolism, 86(3), 1306-1312, 2001);
(24) The methanol extract of the plant Saururus chinensis identified by Kim et al. (Biol. Pharm. Bull., 26(4),481-486,2003) which inhibits the nuclear activation of NFκB by stabilizing cytosolic IκB-α in RAW 264.7 mouse macrophages;
(25) N-acetylcysteine, which reduces NFκB nuclear activation that is induced by hypoxia in mouse embryonic fibroblasts by specifically inhibiting NFκB binding to DNA and thereby inhibiting hypoxia-induced increases in the anti-apoptotic gene product, XIAP (Qanungo et al., J. Biol. Chem., 279(48), 50455-50464, 2004);
(26) Phenylmethyl benzoquinone derivatives, which are shown to inhibit the production of inflammatory mediators and the activation of NF-κB (U.S. Pat. No. 6,943,196);
(27) Xanthine derivatives, which are shown to inhibit NF-κB activation (Japanese Unexamined Patent Publication (kokai) No, 9-227561);
(28) Isoquinoline derivatives, which are shown to inhibit NF-κB activation (Japanese Unexamined Patent Publication (Kokai) No. 10-87491);
(29) Indan derivatives, which are shown to inhibit NF-κB activation (U.S. Pat. No. 6,734,180);
(30) Alkaloids originated from a plant belonging to the genus Stephania of the family Menspermaceae, and their derivatives and salts thereof, which are shown to inhibit NF-κB activation (U.S. Pat. No. 6,123,943);
(31) Agents that inhibit NF-κB activation by modulating the ubiquitin degradation pathway, such as peptides that resemble the recognition domain for E3 ubiquitin ligase (See, e.g., U.S. Pat. No. 6,656,713 and U.S. Pat. No. 5,932,425); and
(32) Antisense oligonucleotide which hybridizes to NFκB mRNA and thus inhibits NFκB dependent pathways (U.S. Pat. No. 6,498,147).

Combinations of these agents or combinatorial use of more than one agent are particularly preferred and may have significant therapeutic advantages in the treatment of MD. In one aspect, the toxicity or side effects of individual compositions may be reduced or practically eliminated when using lower dosages of individual compositions to achieve the same or better therapeutic efficacy as may be obtained from a larger dose of any one composition. Furthermore, the compositions may block the pathway at multiple points to achieve greater therapeutic effects.

An additional benefit of using these compositions is that of improving various qualities of muscular quality. One class of improvement is that of morphology of dystrophic skeletal muscles, particularly in dystrophic muscles that are exposed to chronic passive stretch. The compositions particularly improve the sarcomeric organization of dystrophic muscles and increase the survival of striated muscle fibers in dystrophic muscles by opposing those pathogenic mechanisms responsible for the streaming of Z lines in dystrophic muscle (Cullen, M. J., Fulthorpe, J. J., 1975. Stages in fibre breakdown in Duchenne Muscular Dystrophy: An electron-microscopic study. J of the Neurol. Sci. 24, 179-200.). The compositions may also improve muscle fiber cross sectional diameter, increase the number of muscle nuclei per fiber cross section, and reduce the percentage of centrally located nuclei. A second class of functional improvement is in resting membrane potential, particularly in dystrophic muscles that are exposed to passive muscle stretch. Additionally, there is improvement in whole body strength, as determined by measuring the total body strength exerted by mice exhibiting muscular dystrophy as initially described in Carlson et al.( Muscle and Nerve, 13:480-84,1990) and an improvement in the development of tension in the limb musculature.

Compositions disclosed herein may be administered alone or in combination with, for example, other NFκB inhibitors, steroids, anesthetics, antiepileptics, other agents that affect gene expression, or combinations thereof.

Compositions disclosed herein may be administered, for example, parenterally, to a subject diagnosed with dystrophin deficiency or muscular dystrophy, either by intermittent or continuous intravenous administration or by injection in the muscles. Administration can be given either through a single dose or a series of divided doses. Compounds in various formulations of pharmaceutically effective amounts for treating MD may be used in combination or sequentially and may be administered by intermittent or continuous administration via implantation of a biocompatible, biodegradable polymeric matrix delivery system, via a subdural pump inserted to administer compounds directly, or by intranasal, oral, or rectal administration.

It is desirable to monitor the physiological and morphological changes before and after administration of the compositions disclosed herein. The expression levels of NFκB and IκB in the cells may be measured at both the mRNA and protein levels by Northern blot and Western blot analysis. The activation of NFκB may be measured using the Trans AM assays or other methodology known to artisans in the field.

Blood or tissue samples may be taken from subjects treated with the disclosed compositions to measure the expression profiles of various cytokines as a result of the treatment. Gene expression profile may be analyzed by microarray, RT-PCR, Northern blot, Western blot or by ELISA analyses. Preferably, the levels of TNFα, IL-6 and IL-1β are periodically monitored to assess treatment efficacy.

Changes or modifications may be made in the methods and systems described herein without departing from the spirit hereof. It should thus be noted that the matter contained in the above description or shown in the accompanying figures and examples should be interpreted as illustrative and not in a limiting sense.

Various references, including patents, patent applications and other scientific literatures are cited throughout this application. Full citations for the scientific literatures can be found at the end of the Specification immediately proceeding the Claims. All references are expressly incorporated by reference into this application in order to more fully describe the state of the art.

### EXAMPLE 1

### PDTC May Stabilize Cytosolic IκB-α in Adult mdx Skeletal Muscle

Previous studies have shown that PDTC reduces nuclear NFκB activation (Cuzzocrea et al., 2002, D'Acquisto et al., 1999, D'Acquisto et al., 2001, Rangan et al., 1999, Satoh et al., 1999 and Takeuchi et al., 2002) by stabilizing cytosolic IκB-α (Cuzzocrea et al., 2002) in various tissues from mice and rats. To determine if the doses used in the present study have similar mechanisms of action in adult mdx skeletal muscle, mdx mice were administered either a single ip dose of 50 mg/kg PDTC or vehicle (HEPES Ringer) prior to euthanization and isolation of the diaphragm muscle. Western blots of cytosolic IκB-α obtained at 3 and 5 h after vehicle injection indicated ambient levels of the inhibitory protein in freshly isolated diaphragm muscle (FIGS. 1a and b). A single injection of PDTC substantially increased ambient levels of cytosolic IκB-α at corresponding time points in littermate diaphragms (FIGS. 1c and d). These results indicate that a single dose of PDTC may substantially increase ambient cytosolic IκB-α levels in adult mdx skeletal muscle for a period of at least 5 h.

### EXAMPLE 2

### Chronic PDTC Administration May Reduce the Loss of Striated Fibers and Have Beneficial Effects on the Structure of mdx TS Muscle

Previous studies indicated a significant loss of muscle fibers in the adult mdx TS that did not occur in the nondystrophic TS and that was characterized by an overall 45% reduction in the thickness of the TS based upon the number of fiber layers seen in cross section (Carlson et al., 2003). In contrast to nondystrophic TS fibers which were uniformly striated (FIG. 3), surviving adult mdx TS fibers exhibited discrete cytoplasmic areas devoid of myofibrillar material, areas of hypercontraction, large areas of cytoplasmic rarefaction with delta lesions, and areas of apparent myofibrillar degeneration. By about 2 years of age, the mdx TS appeared as a thin (about 50-100 μm thick) fibrous layer with only a few muscle fibers that lacked myofibrillar organization. Large areas devoid of muscle fibers were characterized by extensive fibrosis with collagen fibrils and numerous fat cells at least as early as 5 months and progressing throughout the life of the mouse (Carlson et al., 2003).

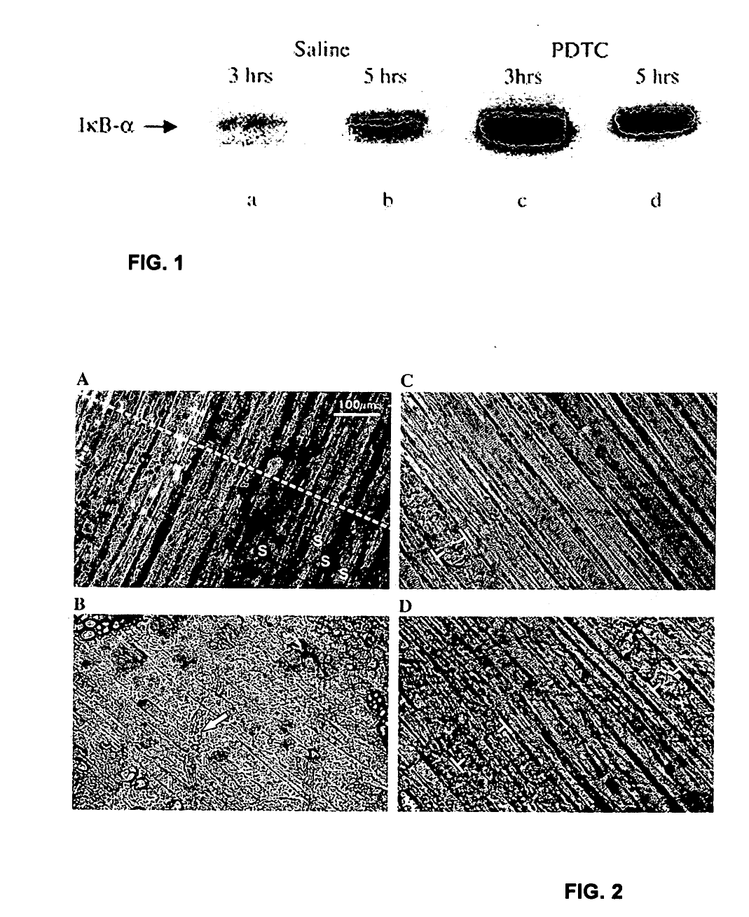

In freshly isolated preparations of mdx TS muscle, hypercontraction and myofibrillar disorganization are expressed as a decrease in the appearance of striated muscle fibers (See e.g., FIG. 2A) and fiber loss and fibrosis appear as a decrease in the number of fibers in individual areas of the TS (See e.g., FIG. 2B; cf. Carlson et al., 2003 and Cullen and Fulthorpe, 1975). In order to quantify these observations and rapidly assess large areas of the mdx TS muscle, several photomicrographs were obtained over large portions of each TS muscle and the proportion of each microscopic field exhibiting muscle fibers and striated muscle fibers was determined for each area sampled. Nondystrophic muscles examined using this technique exhibited uniform levels of muscle fibers and uniform levels of striated muscle fibers throughout the TS muscle (FIG. 3).

The effect of aging on untreated mdx TS muscle is apparent by comparing FIGS. 2A and B, which represent the middle region of the TS muscle at 9 months and 15 months, respectively. Although the untreated 9-month mdx TS muscle exhibited muscle fibers across the entire photographed area, only a small percentage (22%) of these fibers were striated (FIG. 2A). In contrast, the 15-month preparation exhibited only a few muscle fibers (<10%), and no striated fibers at the same magnification over the middle region of the TS muscle (FIG. 2B). This comparison demonstrates the fiber loss that normally occurs in the mdx TS muscle and emphasizes the experimental utility of this particular preparation in directly assessing the damaging effects of chronic passive stretch on dystrophic muscle fibers.

In the first series of PDTC experiments, mdx mice aged 8.5-9 months at sacrifice received daily injections of PDTC at doses between 50 and 75 mg/kg for a period of 27-30 consecutive days. FIG. 2C shows the effects of this treatment on the middle region of the TS muscle. In contrast to the area from an untreated mouse (FIG. 2A), the PDTC-treated mdx TS exhibited approximately 82% striated fibers (FIG. 2C). The combined results from this first series of experiments indicated that both untreated and PDTC-treated preparations exhibited an average percent fibers of about 80%, but that the PDTC-treated preparations had a highly significant (P<0.01) 3-fold increase in the percentage of striated muscle fibers (FIG. 4). These results prompted additional studies using older mdx mice.

In the second series of experiments (Series 2 investigations), mdx mice aged 9.5-16 months at the beginning of the experiment and 11.5-18.5 months at sacrifice (average age at sacrifice for all saline and PDTC-treated mice in this series was 14.8 months) were treated with daily injections of 50 mg/kg PDTC. In this and subsequent experiments, the percentage of muscle fibers and of striated muscle fibers were evaluated in 3 regions of the TS; the caudal third extending towards the xiphoid process, the middle third, and the cephalad third of the muscle. Control experiments using these procedures on adult nondystrophic TS muscles (N=37 areas, 2 TS muscles at 19 and 27 months) yielded average values of 99.1±0.5 (SE) percent fibers and 96.7±1.2 percent striated fibers with no regional differences across the caudal, middle and cephalad thirds of the muscle (FIG. 3). A third series of experiments using daily 50 mg/kg PDTC injections (Series 3 investigations) was conducted using aged mdx mice which were 19-20 months old at the beginning of the experiment and 21.5-22 months old at sacrifice (Series 3). Littermate or age-matched saline-injected mdx mice served as controls.

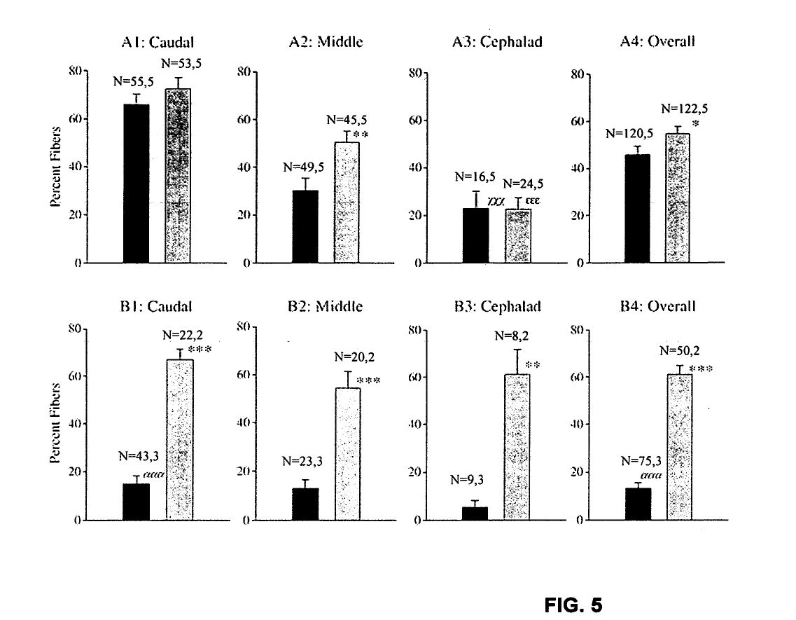

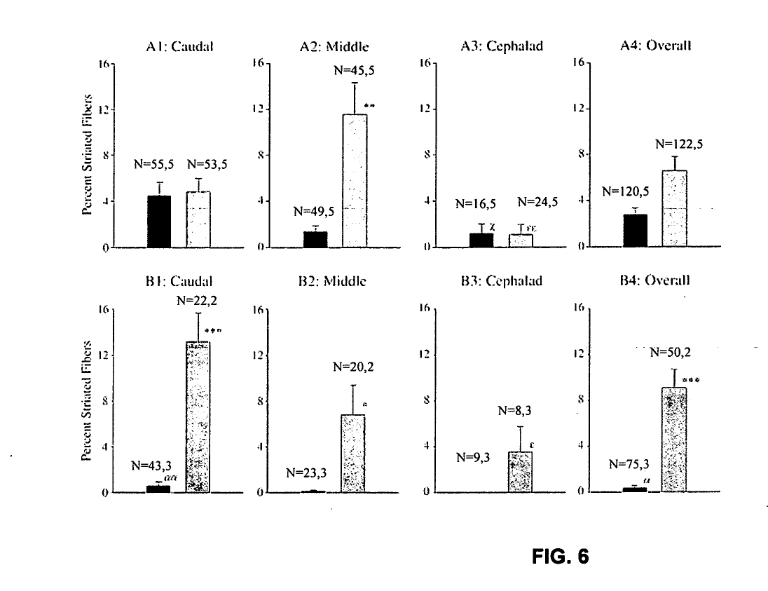

The Series 2 TS muscles exhibited a significant (P<0.001) effect of TS region on the percentage of fibers in both the saline-injected and PDTC-treated preparations (FIG. 5A). In each case, the percent of fibers declined progressively in the cephalad direction. PDTC treatment produced a significant (P<0.01) 1.7-fold increase in the percent of fibers in the middle (compare FIGS. 2B and D) third of the muscle (FIG. 5A2), and a significant increase in the overall percent of fibers across all regions of the muscle (overall; P<0.05). A significant effect of region on the percent of striated fibers was also observed for the Series 2 saline-injected mice at an average age of 14.6 months (P<0.05; FIGS. 6A1-A3). The PDTC-treated mice at this age exhibited a large (8.4-fold) and significantly (P<0.01) increased percent of striated fibers in the middle region (compare FIGS. 2B and D) of the TS muscle (FIG. 6A2).

There was a significant effect of age (between Series 2 and Series 3) on the percent of fibers (FIG. 5B) and striated fibers (FIG. 6B) in the saline-injected mice; and at 22 months, an average of less than 20% of each sampled area contained fibers (black bars in FIGS. 5B1-B4). The decline in fibers with age was highly significant (P<0.001) in the caudal region (compare FIGS. 5A1-B1) and in the overall total of all areas sampled across the TS (compare FIGS. 5B4-A4) but did not reach statistical significance in the middle or cephalad regions. At 22 months, the percent of striated fibers was less than 1% in all three TS regions of the saline-injected mice (FIGS. 6B1-B3; black bars) and was significantly decreased compared to Series 2 in the caudal region (FIG. 6B1; P<0.01) and in the overall total of all sampled areas (FIG. 6B4; α, P<0.05). At 22 months, no significant regional effect was observed for either the percent of fibers (FIG. 5B) or striated fibers (FIG. 6B) in the saline-injected (vehicle) mice. These results indicate that fiber loss and loss of striations in the mdx TS initially occur in the cephalad and middle regions and proceed caudally with aging (FIG. 5 and FIG. 6).

PDTC treatment of the aged mdx mice (22 months) produced large (4.1- to 11.1-fold) and significant increases in the density of muscle fibers across all regions (FIGS. 5B1-B4; P<0.01 or P<0.001) and substantially increased (22- to 68-fold) the percent of striated fibers in the caudal (FIG. 6B1; P<0.001), middle (FIG. 6B2; P<0.05), and overall total of all sampled areas (FIG. 6B4; P<0.001). The PDTC-treated mice at this age did not exhibit a significant effect of region on the percent of fibers (FIGS. 5B1-B3) but did exhibit a significant effect of region (P<0.05) on the percent of striated fibers, with a progressive decline in this value proceeding in the cephalad direction (FIGS. 6B1-B3).

A fourth series of experiments was conducted using a longer period of PDTC administration beginning in younger 5 month mdx mice. The results of these experiments were similar to those in Series 1 through 3 and indicated a significant effect (P<0.001) of PDTC treatment on the percent of striated fibers in both middle and cephalad regions. In particular, treatment of an individual mdx mouse with injections (50 mg/kg) in 238 out of a total of 258 days resulted in an overall percent striated fibers of 40.4±2.6 (N=25 areas) and an overall percent fibers of 82.2±2.6 (N=25) at 13.5 months of age. Since saline-injected mice at an average age of 14.6 months exhibited an overall percent of striated fibers of approximately 2.8±0.6% (FIG. 6A4) and an overall percent fibers of 45.7±3.4%, this result provides strong evidence that PDTC treatment at earlier ages may at least partially prevent the loss of striated fibers in chronically stretched dystrophic muscle. Overall, these results indicate that PDTC treatment reduced the loss of striated muscle fibers by inhibiting and partially reversing a pathogenic mechanism that normally proceeds in the cephalad to caudal direction in the untreated mdx TS muscle.

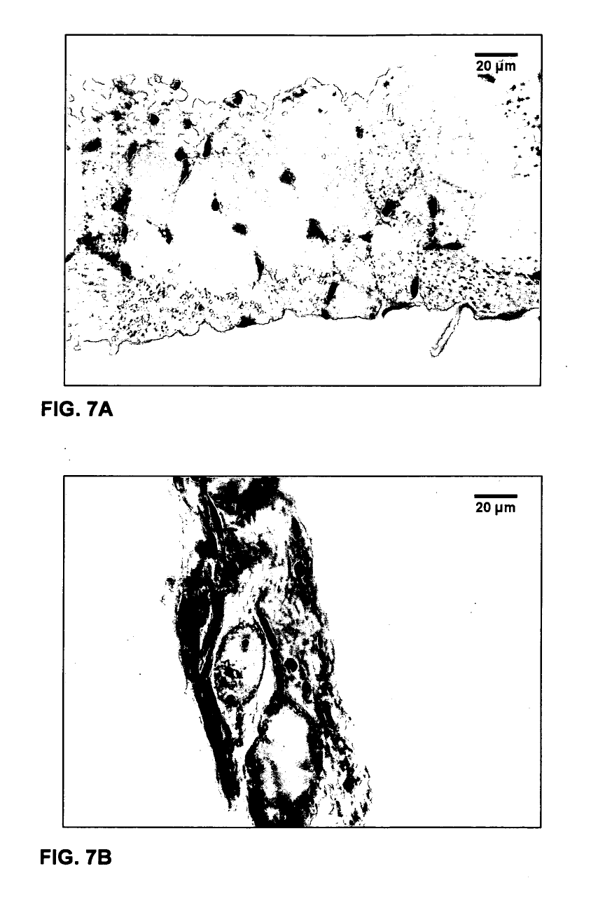

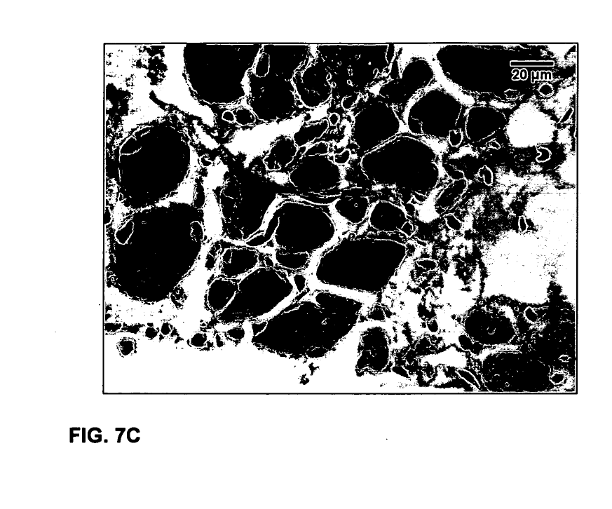

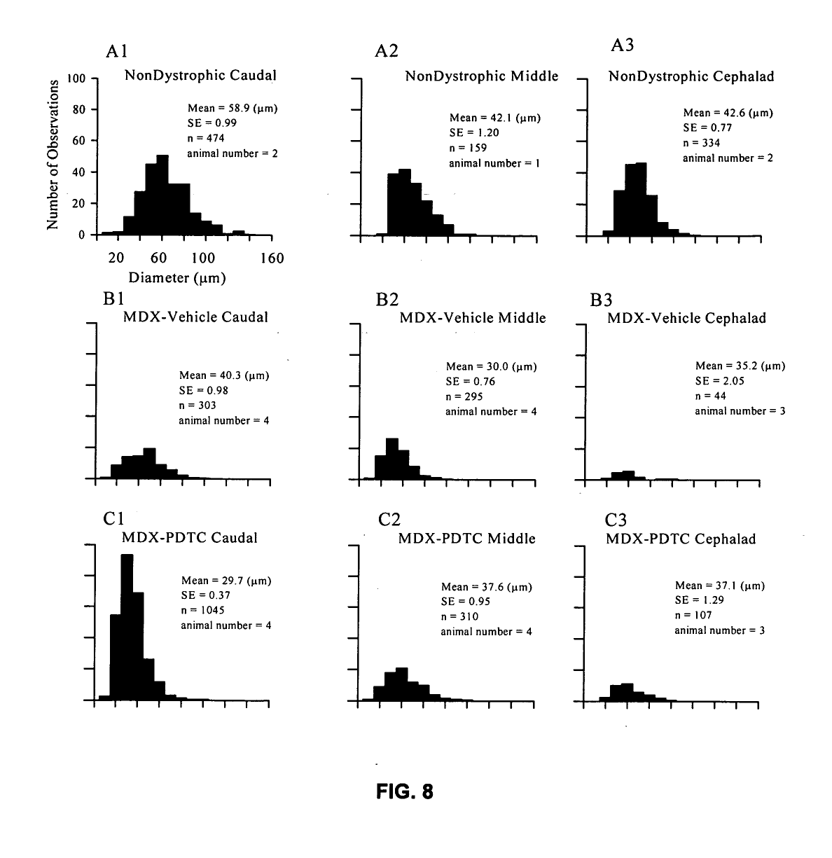

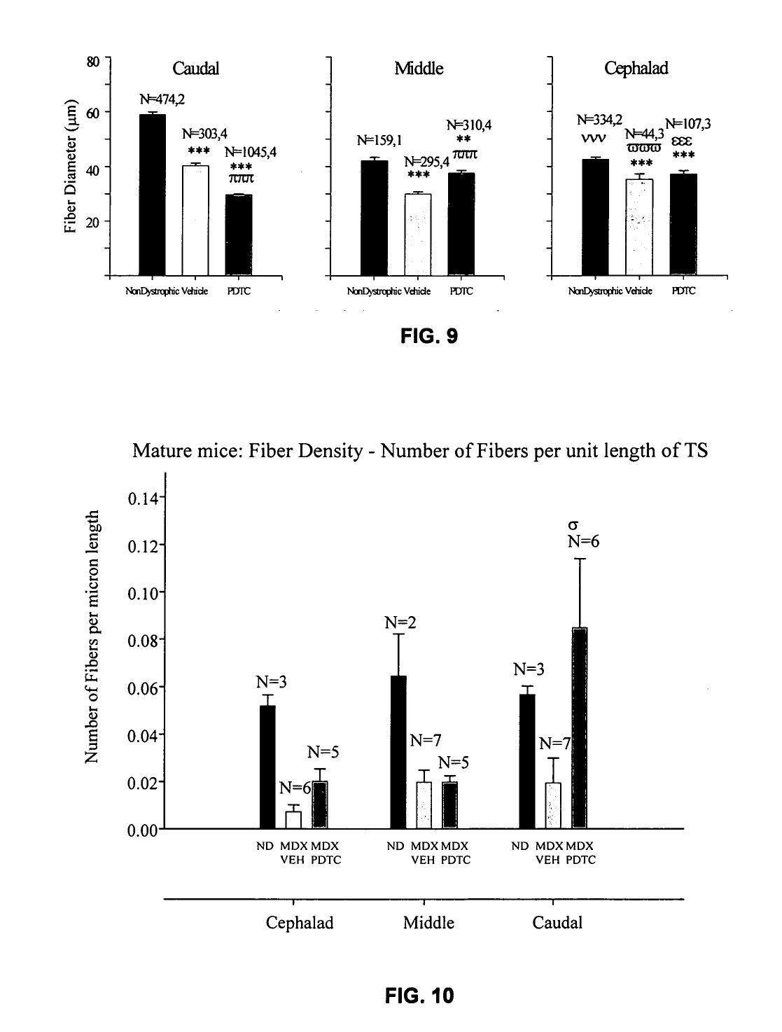

To further examine the morphological effects of chronic treatment with inhibitors of the NFκB pathway, the fixed TS preparations used in these investigations on mature mdx mice (Series 2 through 4) were sectioned and stained with hematoxylin and eosin using standard histological procedures (FIG. 7). The results indicate that there is a total of approximately 500 to 600 fibers in the entire nondystrophic TS (FIGS. 8A1 to A3). Vehicle treated mdx TS muscles from the Series 2 and 3 PDTC experiments exhibited a 68% reduction in fibers in the caudal region, a 54% reduction in the middle region, and a 91% reduction in the cephalad region (FIGS. 8B1 to B3). Results from age-matched PDTC-treated mice indicated substantial increases in fiber number in the caudal region (FIG. 8C1), no change in the middle region (FIG. 8C2), and an increase in fiber number in the cephalad region (FIG. 8C3). All TS preparations (nondystrophic, mdx vehicle, and mdx PDTC treated) exhibited a significant (p<0.001) effect of region on fiber diameter (FIG. 9), a result which suggests that different magnitudes of passive stretch may differentially influence signaling pathways controlling fiber hypertrophy. Vehicle-injected mdx TS fibers exhibited a significant (p<0.001) reduction in diameter in all regions of the TS muscle in comparison to nondystrophic preparations (FIG. 9). PDTC treatment produced a significant (p<0.001) reduction in diameter in the caudal region and a significant (p<0.001) increase in diameter in the middle region in comparison to corresponding values from the vehicle-injected mdx mice (FIG. 9).

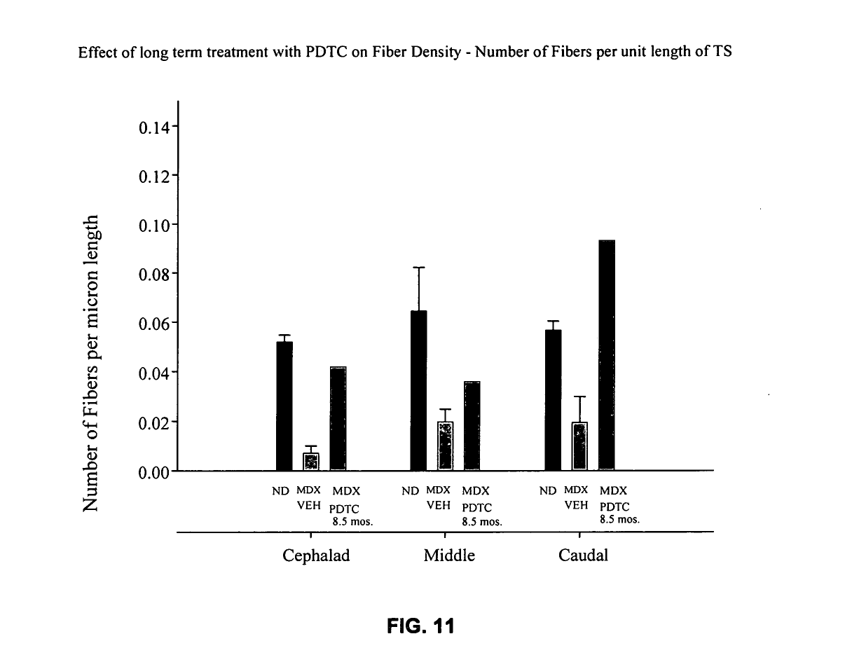

To determine the density of muscle fibers in the mdx TS muscle, individual sections representing all of the fibers within each TS muscle were evaluated by determining the average number of fiber cross sections per unit length of the TS muscle where the length is defined along the axis orthogonal to the principal fiber axis. This procedure was performed for all of the available TS muscles initially used in the Series 2 and 3 PDTC investigations. Mdx TS muscles treated with vehicle exhibited substantial decreases in fiber density in comparison to nondystrophic muscle and treatment with PDTC increased the number of fibers per unit length in both the cephalad and caudal regions (p<0.05) of the mdx TS muscle (FIG. 10). In particular, treatment of an individual mdx mouse with injections (50 mg/kg) in 238 out of a total of 258 days beginning at an age of 5 months and ending at 13.5 months of age resulted in substantial improvements in the density of fibers in comparison to 14.6 month vehicle treated mdx mice (FIG. 11).

These results indicate that treatment of mature mdx mice with PDTC increases fiber number and density (FIGS. 8, 10) and reduces fiber diameter (FIG. 9) in the caudal TS region. Chronic PDTC treatment increased fiber diameter (FIG. 9) without altering fiber number or density (FIGS. 8 and 10) in the middle region of the TS. Each of these effects increases the total cross sectional diameter of working skeletal muscle and is therefore beneficial for dystrophic muscle structure and function. However, the differential effects of the chronic PDTC treatment on fiber diameter and fiber number may have important implications regarding satellite cell proliferation and fusion in dystrophic muscle. One explanation is that the signaling environment in the caudal dystrophic TS exposed to NFκB inhibitors favors the continued fusion of satellite cells into newly regenerated fibers leading to fiber splitting, increased numbers of fiber cross sections, and reduced fiber cross sectional diameters; while the environment in the middle TS favors fusion into a few relatively mature fibers and subsequent activation of pathways promoting differentiation and hypertrophy. These results indicate a therapeutic benefit of chronically inhibiting the NFκB pathway that may be facilitated by adjunctive treatment with inhibitors of other cell signaling pathways in order to compensate for differences in cell signaling which may depend upon the different functions of various dystrophic skeletal muscles.

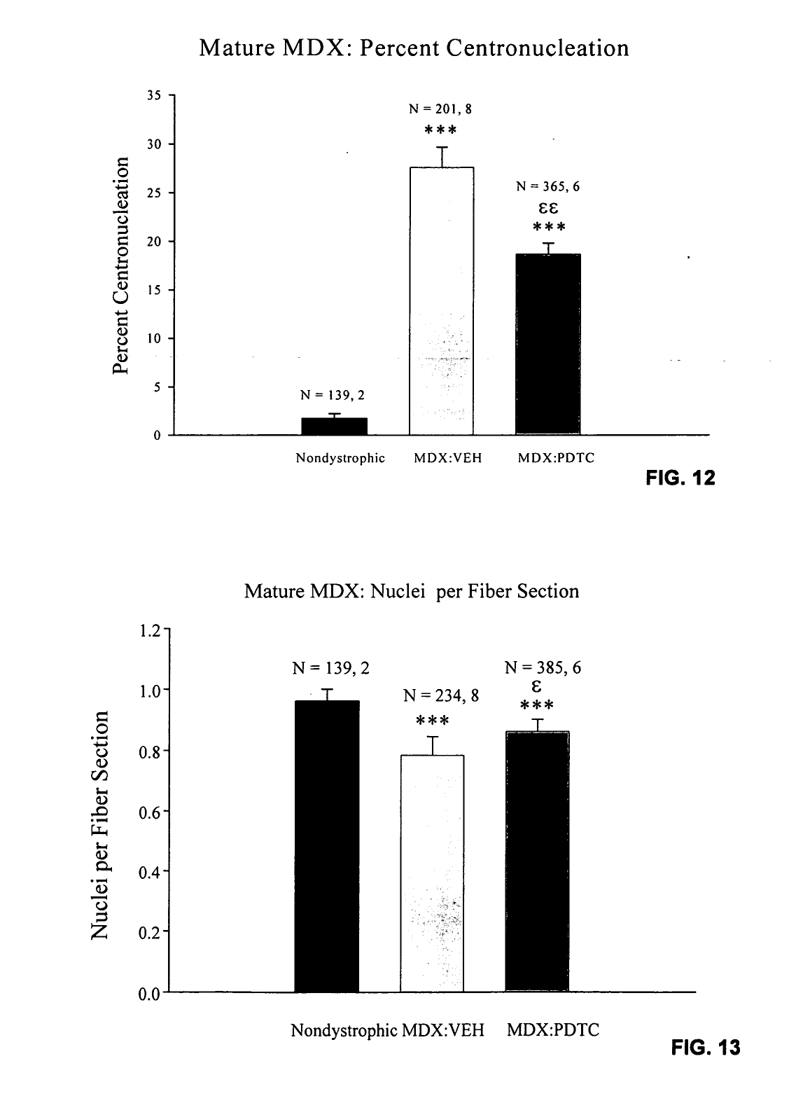

Determination of the total number of myonuclei across all regions of the mature nondystrophic, mdx-vehicle, and mdx-PDTC treated TS muscles in conjunction with determination of the total number of fiber cross sections in the same preparations provided an immediate measure of the number of myonuclei per sectioned fiber (FIG. 12). The results from the Series 2 and 3 PDTC investigations on mature mdx mice indicated that vehicle-injected mdx preparations exhibited a significant (p<0.001) decrease in the number of myonuclei per fiber and that PDTC treatment resulted in a significant (p<0.001) increase in the number of myonuclei per fiber in comparison to age matched vehicle treated mice. Percent centronucleation was increased (p<0.001) in the mdx-vehicle treated TS muscles in comparison to corresponding nondystrophic muscles, and PDTC treatment significantly (p<0.001) reduced percent centronucleation in mature mdx mice (FIG. 13; Series 2 and 3 investigations).

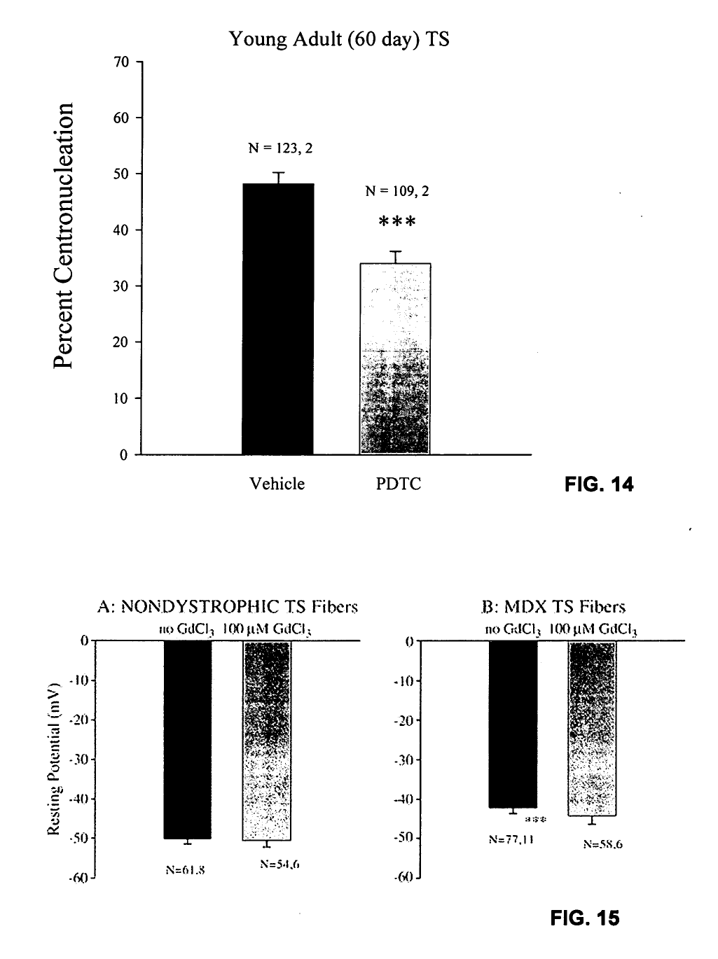

Another demonstration of the beneficial effects of PDTC treatment in reducing the percent centronucleation in dystrophic muscle was obtained by treating 30 day old mdx mice with daily injections of either vehicle or with PDTC (50 mg/kg) for a period of 30 days, and then determining the percent centronucleation at 60 days of age. PDTC treatment for a period of 30 days significantly (p<0.001) reduced the percent centronucleation observed in the young adult (60 day) mdx TS muscles (FIG. 14). These morphological results provide direct evidence that inhibitors of the NFκB pathway may have beneficial effects in treating both young and mature dystrophic preparations.

### EXAMPLE 3

### Mdx TS Muscle Fibers Exhibit Reduced Resting Membrane Potentials that are Not Secondary to Enhanced Divalent Cation Influx

The effect of the Ca²⁺ channel blocker, Gd³⁺, on the resting membrane potential was examined in both nondystrophic and mdx TS muscle preparations. Gd³⁺ blocks both nonselective cation channels and more Ca²⁺-selective leak channels (Franco et al., 1991 and Yang and Sachs, 1989) and, at concentrations of 20-100 μM, eliminates fluorometric determinations of resting Ca²⁺ influx in a variety of cells (Broad et al., 1999, Carlson and Geisbuhler, 2003, Cox et al., 2002 and Samadi et al., 2005). Depending upon the contribution of resting Ca²⁺ influx to the resting membrane potential, addition of blocking concentrations of Gd³⁺ would be expected to hyperpolarize the plasma membrane, and an elevated resting Ca²⁺ influx in mdx muscles would be characterized by an enhanced sensitivity to the hyperpolarizing influence of 100 μM GdCl₃.

The potential effect of Gd³⁺ on resting potential was determined in several nondystrophic (5-17 months) and mdx (5-11.5 months) TS preparations by first recording from several cells in normal HEPES Ringer solution, and then adding 100 μM GdCl₃ to the solution while recording continuously from a single fiber. After removing the electrode from the fiber, the resting potential of several additional fibers were determined in the presence of the lanthanide. The average resting potential in nondystrophic fibers bathed in normal HEPES Ringer was −50.1±1.4 (SE) mV, a value lower than that recorded in this laboratory from nondystrophic diaphragm (−63±1.3 mV, N=98 fibers, 5 weeks to 2 years of age; Carlson and Roshek, 2001). In agreement with previous results from the diaphragm (Carlson and Roshek, 2001), however, mdx TS fibers had a significantly (P<0.001) reduced resting potential of −42.3±1.4 mV (FIG. 15).

No effect on resting potential was observed when Gd³⁺ was added while recording from either nondystrophic or mdx fibers. The results from the total sample of fibers indicated a slight hyperpolarization in the presence of Gd³⁺ that failed to reach statistical significance in either the nondystrophic or mdx TS preparations (FIG. 15). These results are consistent with previous fluorometric studies indicating no significant differences between resting Ca²⁺ influx in nondystrophic and mdx TS muscle fibers (Carlson et al., 2003) and further demonstrate that resting Ca²⁺ influx is not likely to be responsible for the significant resting depolarization that is characteristic of dystrophic muscle fibers (Carlson and Roshek, 2001, Nagel et al., 1990 and Sakakibara et al., 1977).

### EXAMPLE 4

### Chronic PDTC Treatment May Restore or Substantially Improve Resting Membrane Potential in mdx TS Fibers

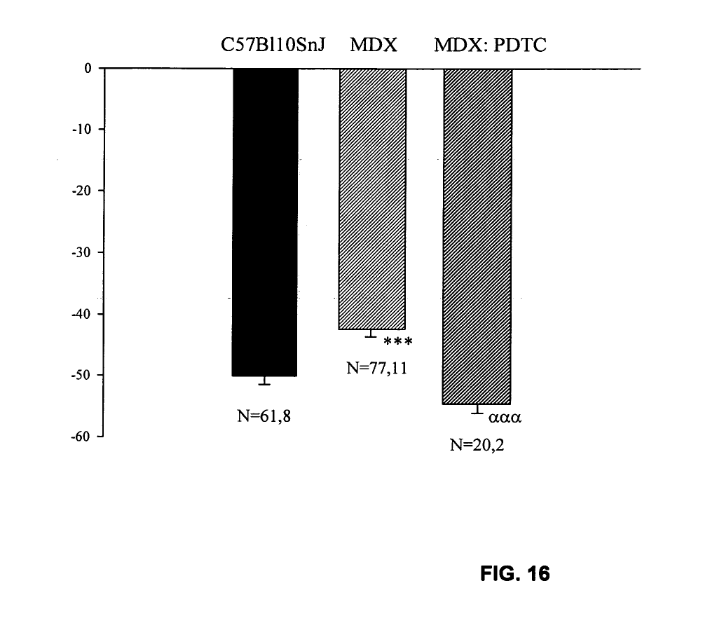

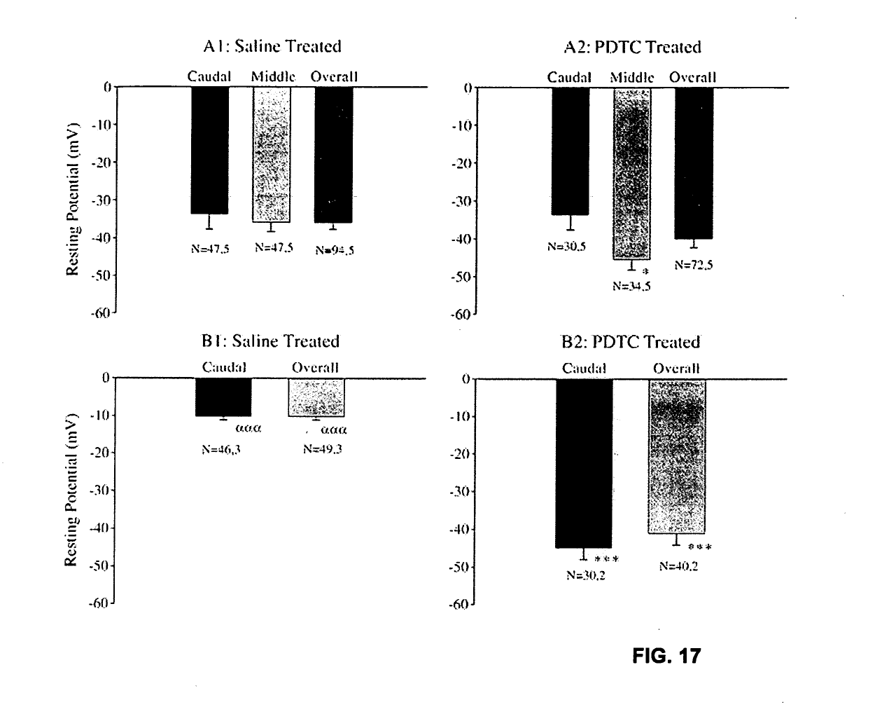

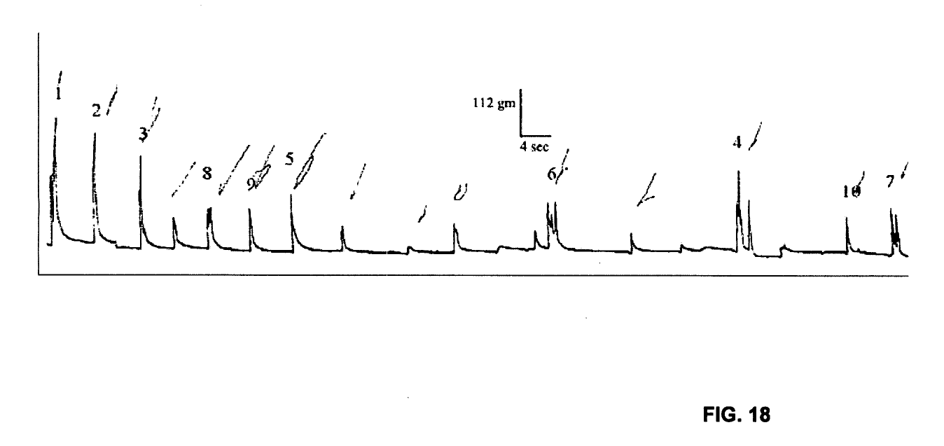

In the Series 1 experiments, daily PDTC treatment beginning at 6.5-7 months completely restored the resting potential to the levels seen in nondystrophic preparations within the middle and caudal regions of the TS (FIG. 16). The mature saline-injected mdx mice used in the Series 2 experiment (12-18 months at sacrifice; average age 14.6 months) exhibited lower resting potentials (overall average=−35.9 mV±1.8 SE) than the younger group of untreated mdx mice used in the initial Gd³⁺ investigations (5-11.5 months; average age 8.2 months). Daily treatment with PDTC increased the fiber resting potential for the older Series 2 mdx mice (FIG. 17A, Series 2) with a significant (P<0.05) increase in the resting potential in the middle region (from −35.8 to −45.4 mV) but not in the caudal region (FIGS. 17A1 and A2).

A significant decrease in resting potential was observed in the aged sample (Series 3) of saline-injected mice (22 months at sacrifice) in comparison to the Series 2 mice (14.6 month average age; FIG. 17B1; P<0.001), with the average resting potential declining to approximately −10 mV. At this age, there were not a sufficient number of intact fibers in either the middle or cephalad regions of saline-injected mice to obtain statistically meaningful determinations from these regions. However, following PDTC treatment it was possible to record from fibers in the middle region of the aged mdx mice (Series 3), where a substantially larger average resting potential of −32.2±6.5 mV (N=9 fibers, 2 mice) was observed (data not shown in FIG. 17). The PDTC-treated mice at this age also showed a highly significant increase in resting potential to approximately −45 mV in the caudal region and −40 mV in the combined data from middle, caudal, and cephalad regions (“overall”) in comparison to their saline-injected littermates (FIG. 17B2; P<0.001).

In the fourth series of PDTC experiments in which chronic administration of the drug was initiated in younger mdx mice (5 months), the overall resting potential obtained at 6.5-13.5 months (49.3±1.4 mV; N=64 fibers, 3 TS muscles) was approximately equal to that observed in nondystrophic TS fibers (−50.1±1.4 mV). In particular, the mdx mouse treated with PDTC between 5 and 13.5 months exhibited an average resting potential at 13.5 months that slightly exceeded nondystrophic levels (−53.3±2.0 mV, N=22 fibers), even though the average resting potential of saline-injected fibers at an average age of 14.6 months was −35.9±1.8 mV (FIG. 17A1). These results show a pronounced beneficial effect of PDTC treatment on the overall health of mdx TS fibers that is consistent with the positive effects of daily PDTC treatment on the survival of striated fibers in the chronically stretched mdx TS.

### EXAMPLE 5

### Chronic Treatment with Inhibitors of the NFκB Pathway May Significantly Reduce an Index of Whole Body Fatigue in the mdx Mouse

FIG. 18 presents individual “forward pulling tensions” (FPTs; upward deflections) recorded from a PDTC-treated mouse (Series 2) using the whole body tension (WBT) technique (Carlson and Makiejus, 1990). The rank order of FPTs from highest to lowest is indicated by the numbers in the figure. The average of the top 5 and top 10 FPTs divided by the mouse body weight are referred to as the WBT5 and WBT10, respectively, and are significantly reduced in mdx mice in comparison to nondystrophic controls (Carlson and Makiejus, 1990). In the Series 2 experiments, WBT measurements were obtained from each of the saline-injected and PDTC-injected mice on days 0, 9, 21 and 40 of the experiment.

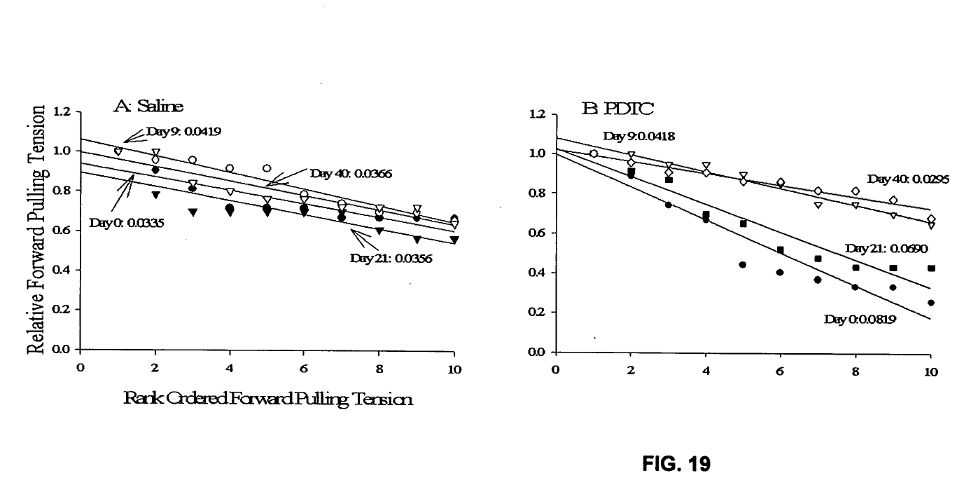

As a first step in examining the potential effects of PDTC treatment on WBT tension development, the slope of the decline in FPTs was evaluated for each recording session for each mouse examined in the Series 2 experiments (FIG. 19). This measure essentially provides a “fatigue index” (FI) since the slope of the decline in FPTs is a direct measure of the average magnitude of the decline in FPT per individual pull for rank-ordered pulls number 2 through 10. If 10 individual pulls obtained during the recording session have identical magnitudes representing the highest FPT, then the FI equals 0. In contrast, a 50% reduction in FPT per pull would produce an FI equal to −0.5.

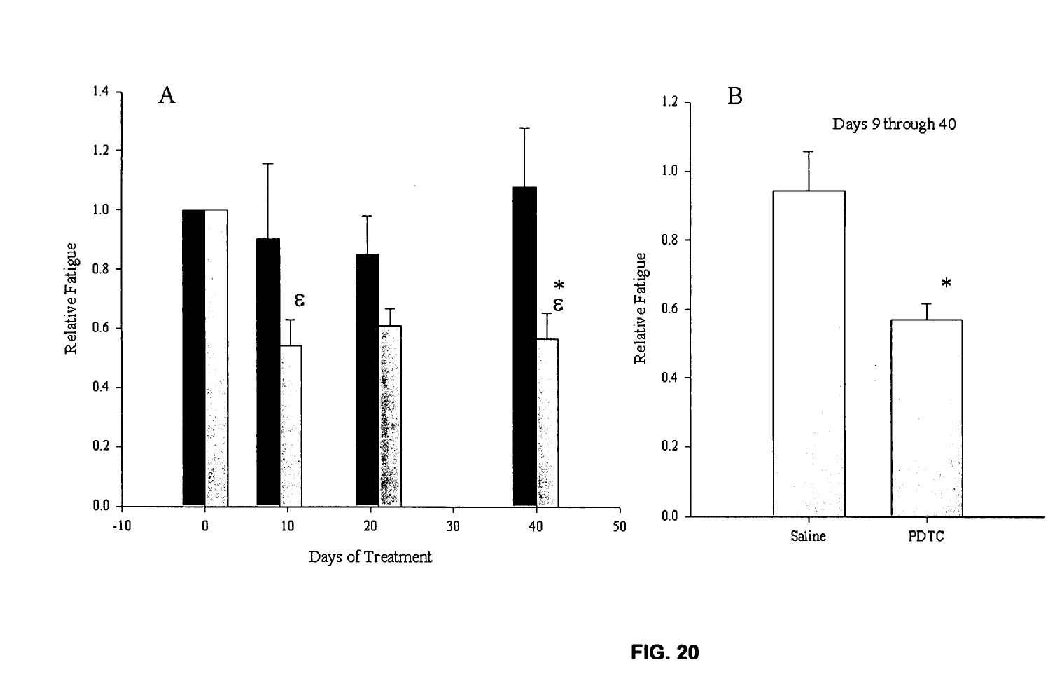

To examine the potential effect of PDTC treatment on FI in age matched adult mdx mice, a pre-treatment FI was obtained during a typical recording session from each of 10 mdx mice used in the Series 2 investigations (age 9.5 to 16 months at beginning of study). Five of the mice were subsequently treated with daily injections of vehicle (HEPES buffered Ringer's solution) and 5 of the mice were treated with daily injections of 50 mg/kg PDTC. Recordings for each of the mice were subsequently obtained on days 9, 21, and 40 of the treatment period (e.g., FIG. 19). To determine the potential effects of treatment on FI, the FIs obtained on days 9, 21, and 40 (e.g., FIG. 19) were each normalized to the pre-treatment FI for each of the 10 mice used in the study. FIG. 20 shows the average normalized FI values for the 5 vehicle-injected and 5 age-matched PDTC-treated mice used in this initial study (FIG. 20A; saline injections-black bars; PDTC injections-gray bars). A decrease in the FI indicating a reduction in the average loss of tension per pull during the course of the study would produce normalized FIs less than one, no change in FI would be associated with normalized FIs equal to 1.0, and increases in FI would produce normalized FIs greater than 1.0.

Daily injections of vehicle did not significantly alter the FI (FIG. 20). In contrast, the PDTC-treated mice showed a significant effect of treatment on the normalized FI (ε, p<0.05; Kruskal-Wallis ranks ANOVA; N=5 mice repetitively sampled) with significant differences observed between Day 0 and Day 9 and between Day 0 and Day 40 (ε, Tukey, p<0.05). Direct comparisons between vehicle-injected and PDTC-injected mice failed to reach significance on Days 9 and 21, but were significantly different (*, p<0.05, t test) at Day 40 of treatment (FIG. 20A). When the post-treatment results (Days 9, 21, and 40) were combined for the two groups (FIG. 20B), there was a significant (*, p<0.05; t test) effect of PDTC treatment in reducing the FI. These results provided the first evidence that chronic treatment with inhibitors of the NFκB pathway produced a mild functional benefit by significantly reducing an index of whole body fatigue in the mdx mouse. It indicates that, during a typical 2 to 3 minute WBT recording session, mdx mice treated with inhibitors of the NFκB pathway exhibit less decline in total body strength and thereby produce more consistent FPTs than do age-matched, vehicle-injected mdx mice.

### EXAMPLE 6

### Chronic Treatment with Inhibitors of the NFκB Pathway Produce Increases in Whole Body Tension in Mature mdx Mice

Carlson and Makiejus (1990) showed that mdx mice as young as 4 to 10 weeks of age exhibit skeletal muscle weakness that can be quantified using a simple noninvasive procedure for assessing whole body strength. In these initial experiments, the top 5 or top 10 FPTs observed during a WBT recording session (FIG. 18) were averaged and divided by the total body weight to obtain noninvasive measures of whole body tension, WBT5 and WBT10, respectively. Mdx mice exhibited significant reductions in WBT5 at all age intervals investigated between 4 weeks and 2 years of age. Although the WBT10 values were not explicitly reported in that initial study, the WBT10/WBT5 ratio was determined as an index of fatigue (“functional reserve”, FR) and shown to be significantly reduced in mdx mice at all age intervals examined (4-10 weeks, 10-20 weeks, >20 weeks).

In the initial series of experiments examining the effects of chronic PDTC administration on WBT in mature mdx mice, 3 separate series of investigations (Series 2, 3, and 4) were conducted using age-matched vehicle-injected and PDTC-injected mice that were older than 5 months of age at the beginning of drug treatment. Series 2 consisted of 10 vehicle-injected and PDTC-injected mice treated initially at 9.5 to 16 months of age. Series 3 mice were treated initially at 19-20 months of age, and series 4 were treated initially at 5 months of age. In each individual series, vehicle and PDTC treated mice were age-matched at the beginning of the study.

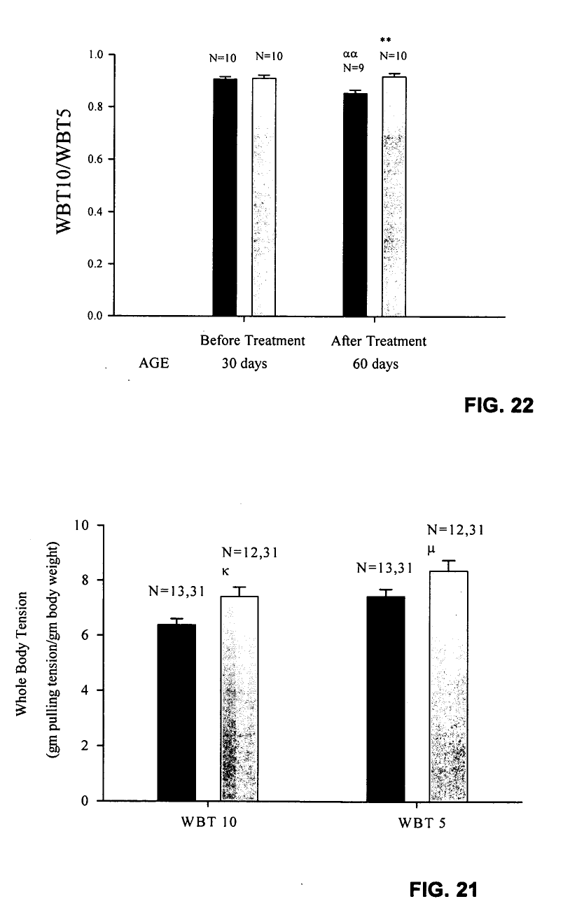

FIG. 21 represents the results from all WBT measurements obtained from all vehicle-injected and PDTC-injected mice in Series 2 through 4. In each case, measurements of WBT5 and WBT10 were obtained prior to the treatment period and on several occasions after at least 20 days of treatment (1 injection per day, cf Carlson et al., 2005). FIG. 21 shows the post-treatment results obtained from all the mice in Series 2 through 4 and indicates that PDTC-treated mice exhibited significantly (p<0.05) elevated WBT10 and WBT5 values in comparison to age-matched, vehicle-injected mdx mice. A comparison of post-treatment to pre-treatment values of WBT10 and WBT5 for vehicle-injected mice indicated no influence of vehicle on these variables, while a similar comparison for PDTC-treated mice showed that chronic treatment with the drug significantly (p<0.05; t test) increased both the WBT10 and the FR above pre-treatment levels. These results indicate that chronic treatment with inhibitors of the NFκB pathway produce a mild functional benefit in increasing total body strength in a manner that is consistent with the reduction in FI and enhanced consistency of FPT seen initially in the Series 2 investigations.

### EXAMPLE 7

### Chronic Treatment with Inhibitors of the NFκB Pathway Prevents Developmental Decreases in Functional Reserve in Young mdx Mice

To further examine the potential functional benefit of treating mdx mice with an agent that stabilizes cytosolic IκBα and inhibits the NFκB pathway, mdx mice at approximately 30 days of age were treated for 30 consecutive days with either vehicle or 50 mg/kg PDTC, and WBT measurements were obtained in each mouse before and after the treatment period. At 30 days of age, mdx mice exhibited a significant reduction in both WBT10 and WBT5 in comparison to nondystrophic mice but had FR (WBT10/WBT5) values that approached those seen in nondystrophic mice. After 30 days of treatment (60 days of age), the vehicle-injected mice showed a significant (αα, p<0.01; t test) age-dependent reduction in FR in comparison to the FR observed in 30 day old mdx mice (FIG. 22). In contrast, mdx mice treated daily with an NFκB inhibitor showed a stable FR of approximately 0.9 that was significantly (**, p<0.01; t test) increased relative to that seen in the vehicle-injected mdx mice at 60 days of age. Although both the WBT10 and WBT5 values were increased relative to the vehicle-injected mice, these increases did not reach statistical significance at 60 days of age. However, the WBT10 value was increased significantly more than the WBT 5 value in the PDTC treated mice, thus producing the significant increase in FR at this age. These results indicate that the FR in mdx mice declines significantly between 1 and 2 months of age and that daily treatment with an inhibitor of the NFκB pathway prevents this decline in muscle function that is a characteristic of Duchenne and related muscular dystrophies.

In summary, chronic treatment of a sample of age-matched mature mdx mice (age 9.5 to 16 months at beginning of treatment period) indicated a significant (p<0.05) improvement in the FI, a measure of the decline in FPT over the top 10 FPTs in the noninvasive procedure for determining whole body tension (Carlson and Makiejus, 1990). This indicates that PDTC-treated mature mdx mice exhibited significant functional improvement characterized by the ability to produce more consistent FPTs over a typical WBT recording session (FIG. 20). Secondly, a larger sample of mature mdx mice exhibited a significant improvement in both WBT5 and WBT10 in comparison to age-matched, vehicle-injected mice (FIG. 21). This indicates that PDTC treatment induced mild but significant increases in total body strength even when administered to mice that were in the advanced stages of the disease. The third observation in these initial studies was that PDTC treatment prevented a decline in the FR when administered to a population of young adult mdx mice (FIG. 22). This indicates that inhibition of the NFκB pathway in young mdx mice prevents a decline in function that is characteristic of Duchenne and related muscular dystrophies. Overall, these results indicate that in vivo inhibition of the NFκB pathway produces a mild functional benefit in increasing total body strength primarily by enhancing the consistency of FPTs in the non-invasive determination of whole body strength (Carlson and Makiejus, 1990).

### EXAMPLE 8

### Chronic in vivo Treatment with Inhibitors of the NFκB Pathway Improves Tension Development in Adult Isolated mdx Muscle Preparations

To further determine whether treatment with NFκB inhibitors influences skeletal muscle function in dystrophic muscle, mdx mice were treated daily with either PDTC (50 mg/kg) or vehicle as previously described (Carlson et al., 2005). In one set of experiments, mature mdx mice (average age 15 months at sacrifice) were treated for at least 2 months (average treatment period=72 days). Following the treatment period, the mice were anaesthetized with sodium pentobarbitol (0.1 mg/kg, ip) and the gastrocnemius preparation (lateral and medial heads, associated plantaris muscle intact) was isolated as initially described in Carlson and Makiejus (1990).

Briefly, the mouse to be examined is placed ventral side down and the gastrocnemius muscle exposed after cutting or reflecting the overlying sartorius muscle. The calcaneous and plantaris tendons are first tied with surgical thread that is tied to a metal hook that fits directly into an isometric tension transducer (Grass Instruments, Model FT03C). After cutting the tendons distal to the ligature, the gastrocnemius, plantaris and soleus muscles are reflected dorsally and the soleus muscle removed from the preparation. The preparation is kept moist with HEPES buffered Ringer solution (in mM: 147.5 NaCl, 5 KCl, 2 CaCl₂, 11 glucose, 5 HEPES, pH 7.35) throughout the surgery and the mouse hindlimb is firmly attached to the surface of a Sylgaard tray using pins (which do penetrate any tissue) or specially constructed hooks. The metal hook and thread are then attached to the isometric tension transducer which is itself attached to a micromanipulator (Narishige) that is used to alter muscle resting length. The muscle is stimulated directly (Grass S9 stimulator) with suprathreshold pulses using 1 inch fine (approximate gauge 26) bipolar silver chloride electrodes that are spaced approximately 5 mm apart on the reflected surface of the muscle preparation. The muscle preparation is mildly stretched while applying 5 to 10 individual pulses (4 msec) of increasing intensity to determine the intensity that produces an asymptotic maximum in twitch tension. The preparation is then stimulated with approximately 10 to 20 individual pulses at this suprathreshold intensity while the muscle length is systematically altered to determine the optimal length for maximal tension development (Iₒ). During this time period and throughout the recording session, the muscle is periodically moistened (about every 2 minutes) with HEPES Ringer and the depth and frequency of respiration is noted. In the rare cases in which the mouse was initially over-anesthetized (<=5% of experiments in practice), a rapid fatigue of tension development was observed signaling a decline in cardiac output during the recording session. Such preparations which did not exhibit stable twitch amplitudes under non-fatiguing stimulation (e.g., 1 pulse 30 seconds) were discarded from further analyses. In rare circumstances, preparations initially exhibited stable twitch amplitudes and subsequently exhibited declining tetanic tensions that suggested a decline in cardiac output. Under these circumstances, the tetanic tension data was discarded from further analyses.

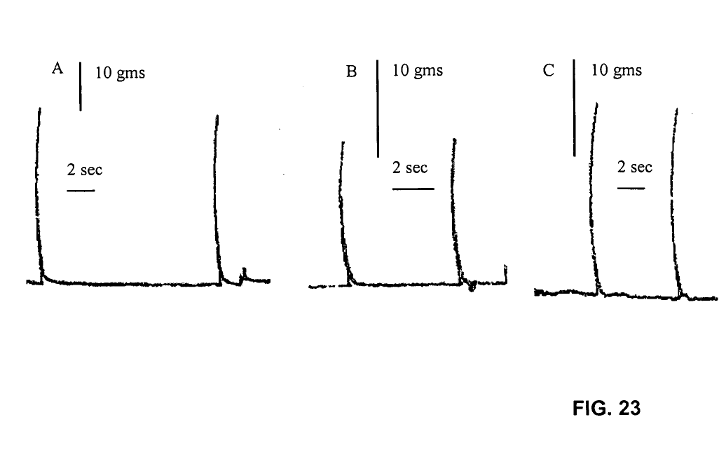

Optimal length was determined by carefully measuring increasing tensions as the muscle length was increased along the ascending limb of the length-tension curve and then noting a 5 or 10% decline in tension as the muscle was lengthened beyond Iₒ. At this point, the muscle length was shortened back to Iₒ and stimulated approximately 5 times (with appropriate rest periods between stimulations) to determine the twitch tension at Iₒ (FIG. 23). After determining twitch tension at Iₒ, the preparation was stimulated briefly (1-2 minutes) at frequencies of 0.2, 0.5, and 1.0 Hz to assess the stability of twitch tension. Stimulation was then applied at 10 Hz for a period of 20 to 60 sec to assess the decline in twitch tension at this frequency. The subsequent rate of recovery of twitch amplitude over a period of approximately 1 to 3 minutes following 10 Hz stimulation was also determined before assessing twitch:tetanus ratios (about 1-2 sec tetanic stimulation) at both 30 and 50 Hz. Finally, the decay of twitch amplitude at 10 Hz and the subsequent recovery of twitch tension was assessed two more times before obtaining plasma samples and euthanizing the animal.

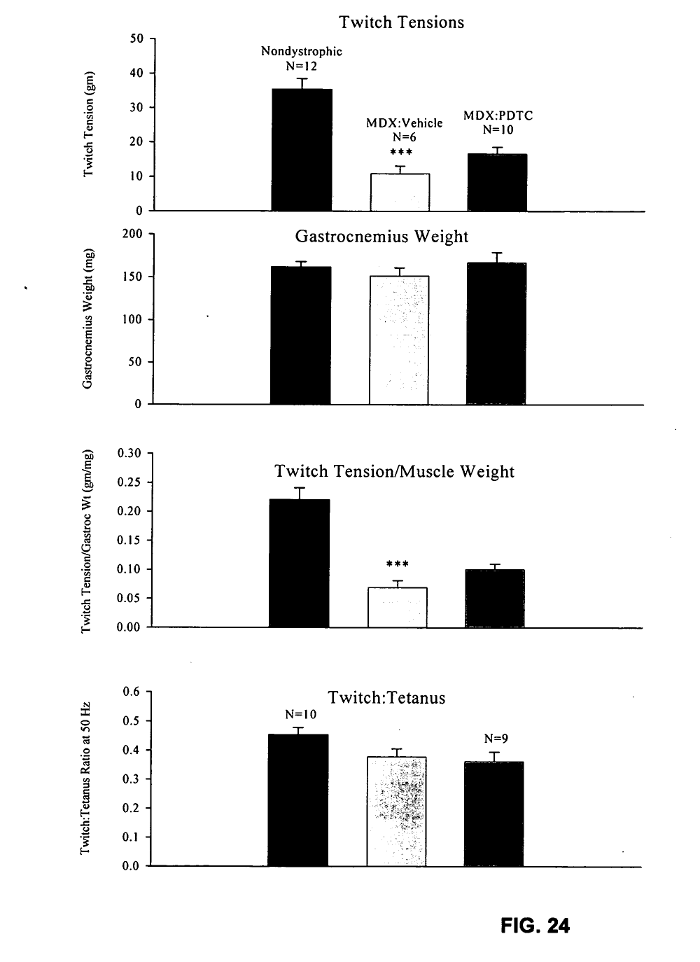

As initially reported in Carlson and Makiejus (1990), in comparison to age-matched nondystrophic mice, mature vehicle-injected mdx mice (average age about 15 months, Series 2-4; cf Carlson et al., 2005) exhibited highly significant (p<0.001) decreases in twitch tension (gm) and specific twitch tension (gm tension/mg muscle wet weight) to approximately 30% of nondystrophic values with no significant changes in gastrocnemius weight or in twitch/tension ratio (FIG. 24). Mature, PDTC-treated mice exhibited a 52% improvement (relative to vehicle-injected mice) in the average twitch tension and a 45% improvement in twitch tension/gm that just failed to reach statistical significance (p>=0.05).

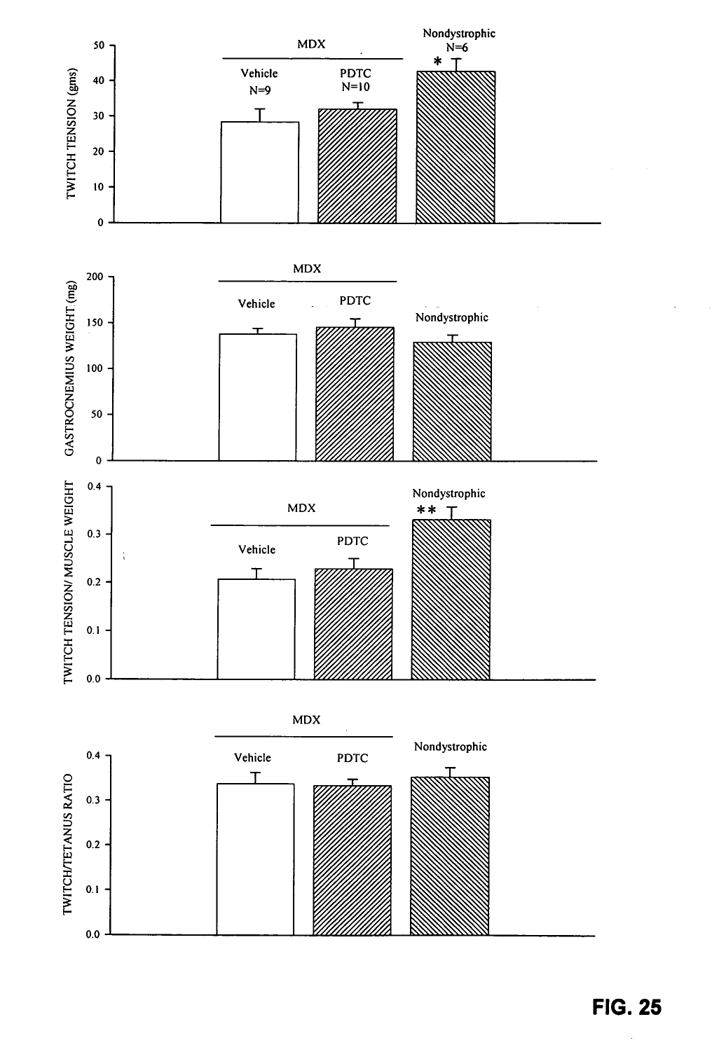

Similar results were obtained from young adult mice (2 month old). In this case, the mdx twitch tensions and twitch tension/gm muscle weight were significantly reduced to 66% (p<0.05) and 62% (p<0.01), respectively, of the corresponding nondystrophic values (FIG. 25). In comparison, the mature (15 month) mdx preparations exhibited twitch and twitch/muscle weight values of approximately 30% of nondystrophic levels (FIG. 24). These results indicate that the mdx gastrocnemius muscle progressively weakens as a consequence of the dystrophic process, similar to what is observed in Duchenne muscular dystrophy. This conclusion indicates that the mdx mouse is a functionally valid model for studying the potential utility of proposed treatments for Duchenne and the related muscular dystrophies (Carlson and Makiejus, 1990). In comparison to mdx vehicle-treated mice, PDTC treatment produced a 13% increase in twitch tension and an 11% increase in twitch tension/mg muscle weight in the young adult preparations (FIG. 25).

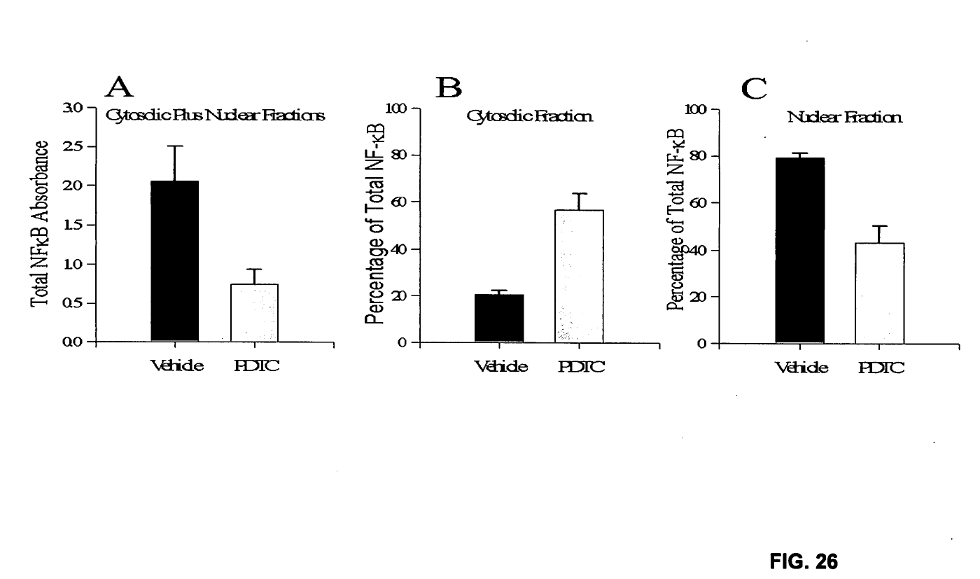

The results of Trans AM assays demonstrate that chronic treatment of adult mdx mice with PDTC reduces total cellular NFκB and the proportion of nuclear NFκB in mdx skeletal muscle (FIG. 26). The Trans AM assay and corresponding electrophoretic mobility shift assays (EMSA), along with Western Blot analyses of IκB-α (FIG. 1) and total cellular NFκB, are useful for screening compounds that inhibit the NFκB pathway in dystrophic skeletal muscle and identifying new compounds that have corresponding beneficial effects in treating dystrophic subjects.

### EXAMPLE 9

### Cytosolic Extracts of Adult mdx Skeletal Muscle Exhibit Significant Increases in Cytokine Expression in Comparison to Nondystrophic Preparations

To determine whether cytosolic extracts from dystrophic muscle exhibit elevated levels of cytokines that are regulated by NFκB, the diaphragm muscles were removed from euthanized mature nondystrophic (C57BI10SnJ) and mdx mice, and cytosolic and nuclear extracts were obtained from this muscle tissue using procedures that were slightly modified from those described in Carlson et al. (Neurobiology of Disease, 20, 719-730, 2005). In these experiments, the nondystrophic mice were between 8 and 31 months of age at euthanasia (average age—18.4 months) and the mdx mice had an average age of 14.6 months. Each diaphragm was divided into costal and crural regions which were processed and analyzed separately. After determining protein concentrations for each of the cytosolic and nuclear extracts (Lowry assay), samples of each extract were used to obtain standard ELISA determinations of mouse TNFα, IL-6, and IL-1β using standard procedures (Assay Designs, Inc.). These particular NFκB products were chosen because of their role in the inflammatory reaction and previously published results indicating the presence of these cytokines in nondystrophic skeletal muscle homogenates (Molina et al., Neuroimmunomodulation, 4, 28-36, 1997; Jonsdottir et al., J. of Physiol. (Lond.), 528.1, 157-163, 2000; Lang et al., Shock, 19(6), 538-546, 2003).

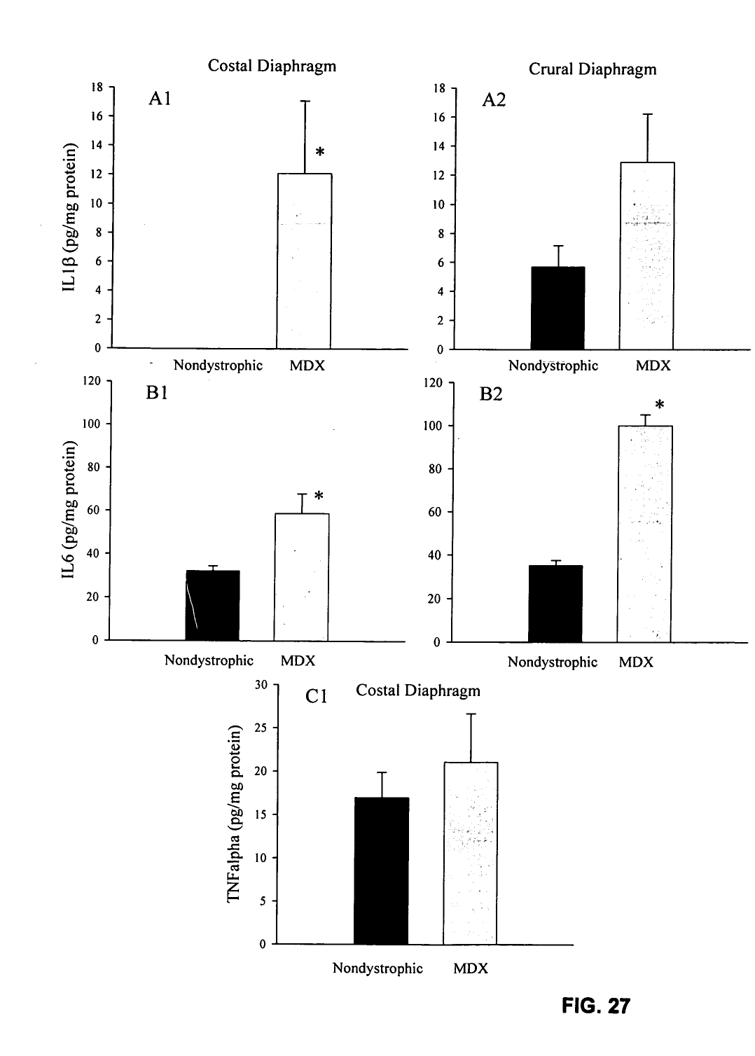

FIG. 27 shows that the cytokines TNFα, IL-6, and IL-1β were each present in conventional cytosolic extracts of skeletal muscle and that freshly excised costal and/or crural diaphragms from mdx mice exhibited statistically significant (p<0.05) increases in IL-1β and IL6 in comparison to corresponding adult nondystrophic preparations. The levels of TNFα were also elevated in the costal mdx diaphragm but this increase did not reach statistical significance (p>0.05).

### EXAMPLE 10

### Sulfasalazine Treatment in vivo May Reduce Skeletal Muscle Nuclear Activation of NFκB in Dystrophic (mdx) Skeletal Muscle

MDX mice aged 3 to 3.5 months were injected with either sulfasalazine (SS; 100 mg/kg, ip) or vehicle (HEPES Ringer; in mM: 147.5 NaCl, 5 KCl, 2CaCl2, 11 glucose, 5 HEPES, pH 7.35) and euthanized at 3 hours post-injection. The costal diaphragms were removed and flash frozen before beginning the extract procedure. Nuclear and cytosolic extracts were obtained using a procedure slightly modified from that described in Carlson et al. (2005) and Kumar and Boriek (2003). Briefly, the muscles were weighed after removing tendinous components, and frozen and homogenized by mortar and pestle in low salt lysis (LSL) buffer on ice (1 mg muscle wet weight/18 μl solution; in mM: 10 HEPES, 10 KCl, 1.5 MgCl₂, 0.1 EDTA, 0.1 EGTA, 1 dithiothreitol (DTT), 0.5 phenylmethylsulfonylfluoride (PMSF); 0.5 mg/ml benzamidine, 4.0 μl/ml protease inhibitor cocktail Sigma #8340 (PIC) to produce the following final concentrations—2.1 μg/ml leupeptin, 3.85 μg/ml aprotinin, 0.416 mM AEBSF (Sigma A8456), 16 μM bestatin, 6 μM pepstatin A, 5.6 μM E64; pH 7.9). To lyse the cells, the ground tissue was subjected to 2 freeze-thaw cycles (5 minute freeze on dry ice followed by thawing at room temperature), and subsequently vortexed and centrifuged (13,000 rpm, 15 sec). The supernatant cytosolic extract was immediately frozen (−80° C.) for subsequent analyses as needed, while the nuclear pellet was washed one time with 500 μl of low salt lysis buffer before being resuspended on ice in a high salt nuclear extraction buffer (in mM: 20 HEPES, 420 NaCl, 1 EDTA, 1 EGTA, 1 DTT, 0.5 PMSF; 25% glycerol, 0.5 mg/ml benzamidine, 4.0 μl/ml PIC; pH 7.9) at a ratio of 4 μl of solution per mg muscle wet weight. The suspension was incubated on ice for 30 minutes and vortexed for 10 sec every 5 minutes before being centrifuged (13,000 rpm, 6 minutes) at 4° C. The supernatant nuclear extract was then removed and frozen (−80° C.) for the subsequent EMSA which was performed within 5 days following the in vivo treatment. Corresponding experiments to determine potential contamination of nuclear extracts with cytoplasmic proteins were performed by determining cytokine levels in both nuclear and cytosolic extracts of dystrophic muscle (ELISA assay). The results of these experiments indicated essentially zero contamination of the nuclear extracts by cytoplasmic proteins. Protein determinations were determined by the method of Lowry using bovine serum albumin standards (20 μg nuclear protein was added to each lane).

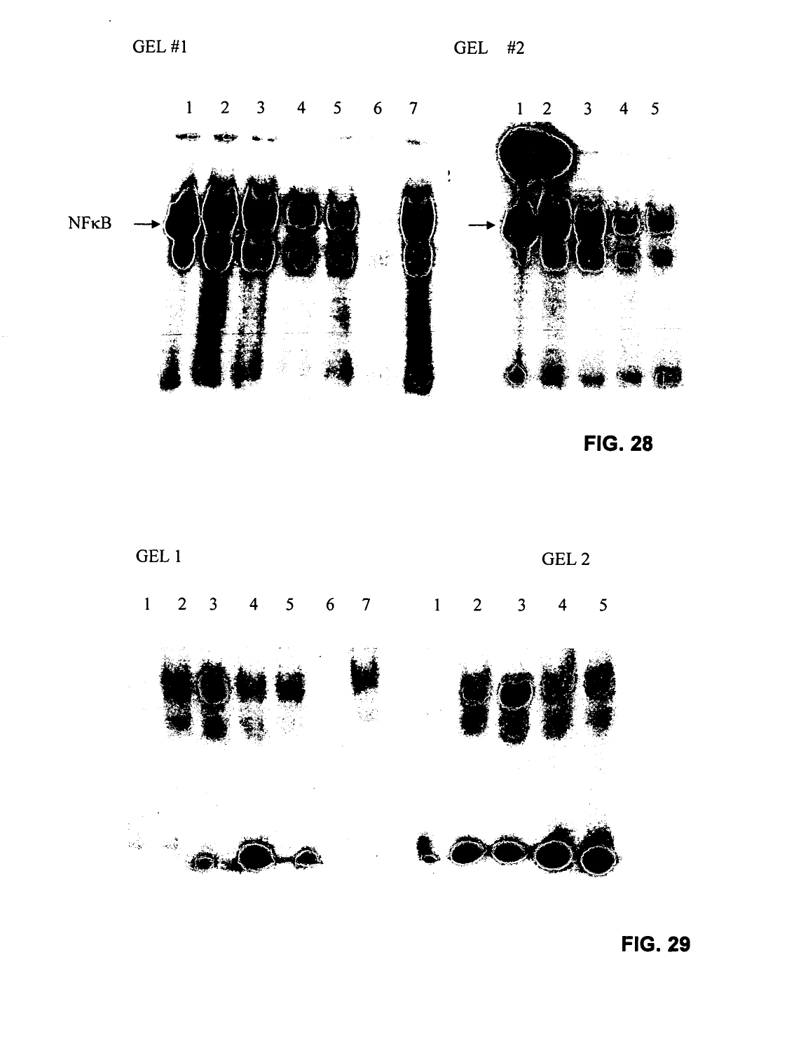

The results shown in FIG. 28 indicate a reduction of nuclear NFκB binding to the appropriate consensus sequence in the SS treated mice (compare lanes 4 and 5 to lanes 2 and 3). Based upon the therapeutic effects of chronic PDTC treatment (Carlson et al., 2005), which reduced total cellular NFκB and the proportion of nuclear NFκB in mdx skeletal muscle (FIG. 26) by stabilizing cytosolic IκB-α (FIG. 1), these results showing an acute effect of SS on the nuclear activation of NFκB in dystrophic muscle directly indicate a therapeutic benefit in treating muscular dystrophy.

### EXAMPLE 11

### Parthenolide Treatment in vivo Reduces Skeletal Muscle Nuclear Activation of NFκB in Dystrophic (mdx) Skeletal Muscle

MDX mice, aged 3 to 3.5 months, were injected with either parthenolide (5 mg/kg in vehicle) or vehicle alone (HEPES Ringer; in mM: 147.5 NaCl, 5 KCl, 2CaCl2, 11 glucose, 5 HEPES, pH 7.35; 0.1% DMSO) and euthanized at 3 hours post-injection. The costal diaphragms were removed and flash frozen before beginning the extract procedure. Nuclear and cytosolic extracts were obtained as described in Example 10. Nuclear extracts were used for EMSA assay as described in Example 10. Protein determinations were determined by the method of Lowry using bovine serum albumin standards (20 μg nuclear protein was added to each lane).

Parthenolide is a member of the class of sesquiterpene lactones that inhibits NFκB activation by a variety of mechanisms including stabilization of cytosolic IκB-α (Hehner et al., J. Biol. Chem., 273(3), 1288-1297, 1998; Wong and Menendez, Biochem. Biophysica Res. Comm., 262, 375-380, 1999), inhibition of IKK (Kwok et al., Chem and Biol., 8, 759-766, 2001), and inhibition of the binding of NFκB to the κB consensus sequence (Sheehan et al., Molec. Pharmacol., 61, 953-963, 2002). The results shown in FIG. 29 were analyzed densitometrically to indicate that the single injection of PTN reduced the nuclear activation of NKκB by 50% in comparison to the vehicle injected dystrophic mice (compare lanes 4 and 5 to lanes 2 and 3). These results provide the first demonstration that parthenolide and other sesquiterpene lactones inhibit the nuclear activation of NFκB in dystrophic skeletal muscle following a single in vivo injection. Based upon the therapeutic effects of chronic PDTC treatment (Carlson et al., 2005), which reduced total cellular NFκB and the proportion of nuclear NFκB in mdx skeletal muscle (FIG. 26) by stabilizing cytosolic IκB-α (FIG. 1), these results showing an acute effect of parthenolide and other sesquiterpene lactones on the nuclear activation of NFκB in dystrophic muscle provide the first direct indication of the therapeutic utility of this class of substances in treating muscular dystrophy.

### EXAMPLE 12

### Sulfasalazine Treatment in vivo May Reduce Cytokine Expression in Some Dystrophic (mdx) Skeletal Muscles

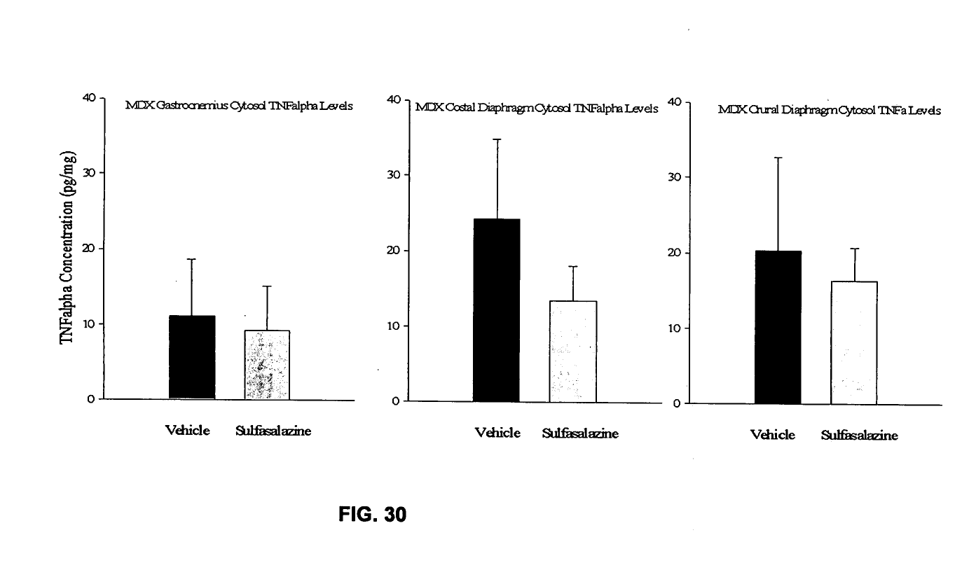

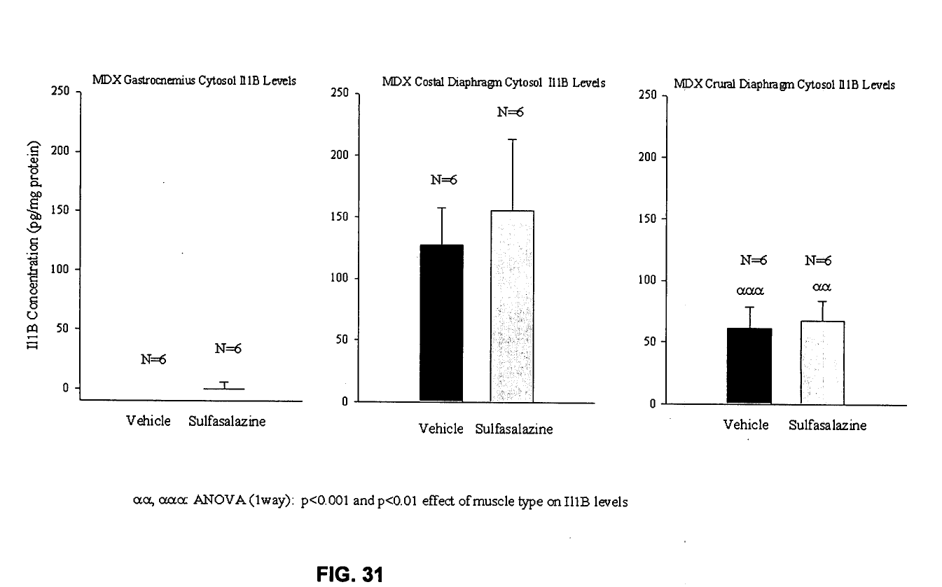

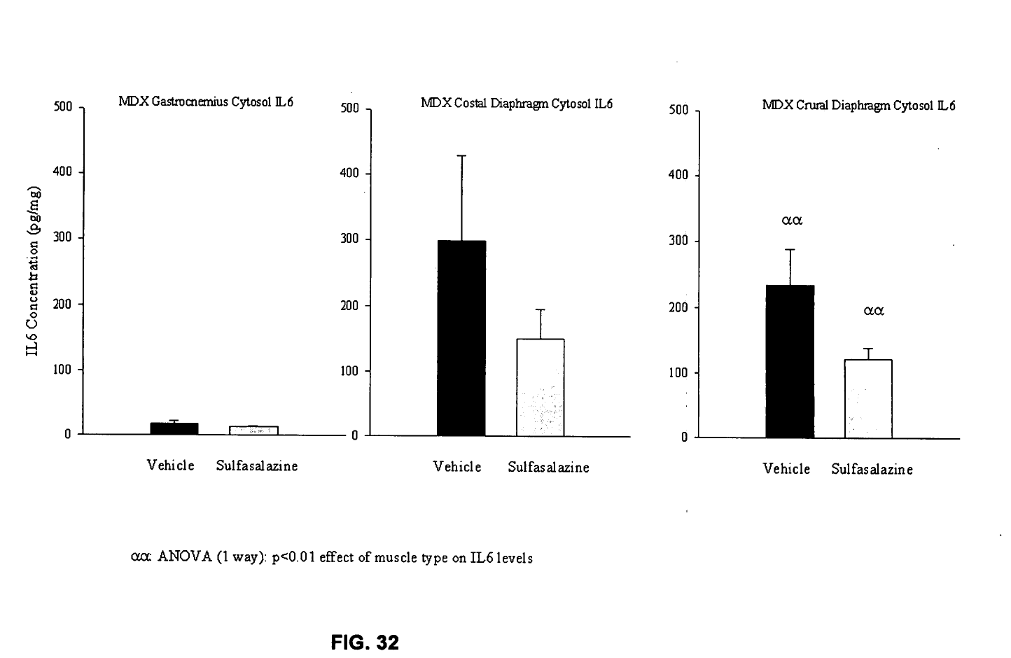

MDX mice aged 3 to 3.5 months were injected with either SS (100 mg/kg) or vehicle (HEPES Ringer; in mM: 147.5 NaCl, 5 KCl, 2CaCl2, 11 glucose, 5 HEPES, pH 7.35) and euthanized at 3 hours post-injection. The costal and crural diaphragms and the gastrocnemius muscle were then immediately removed and flash frozen before beginning the procedure to obtain nuclear and cytosolic extracts as described in Example 10. ELISA determinations of TNFα, II1-β, and IL6 were determined as described in Example 9. FIG. 30 shows that mdx gastrocnemius, costal diaphragm, and crural diaphragms exhibited roughly equivalent expression of TNFα and that a single injection of sulfasalazine reduced the expression of TNFα in the cytosolic extracts from mdx costal diaphragm. FIG. 31 shows that the expression of IL1-β was significantly increased in both the costal and crural diaphragms in comparison to the gastrocnemius muscle which did not exhibit IL1-β expression. This observation suggests that the expression of IL1-β may contribute to the more severe phenotype characteristic of mdx diaphragm muscle. A single injection of sulfasalazine did not reduce the expression of IL1-β. However, a single injection of sulfasalazine did reduce the expression of IL6 in both the costal and crural diaphragms (FIG. 32) suggesting that the significantly (p<0.01) elevated expression of this cytokine in mdx diaphragm in comparison to gastrocnemius may contribute to the dystrophic phenotype. These results are consistent with the results showing that a single sulfasalazine injection reduces the nuclear activation of NFκB (FIG. 28), and further suggest that the potential utility of various compounds in treating muscular dystrophy may be assessed by determining the acute and chronic effects of the compound on cytokine expression in a variety of dystrophic muscle samples.

### EXAMPLE 13

### Chronic in vivo Treatment with Sulfasalazine Significantly Improves the Resting Membrane Potential in Dystrophic (mdx) Triangularis Sterni (TS) Muscle Fibers

Sulfasalazine (“SS”) (Sigma Number S-0883) was dissolved in standard HEPES Ringer solution (pH 7.35) at a concentration of 10 mg/ml by adding a few drops of 0.5 M NaOH until the solution turned a clear pink or red indicating that the sulfasalazine powder had dissolved (pH approximately 10.0). An equal amount of 0.5 M NaOH was then added to an equal volume of vehicle. These solutions were immediately used to inject one sulfasalazine-treated mouse (ip) with 100 mg/kg of the sulfasalazine solution (0.3 ml for a 30 gm mouse) and another littermate with an equivalent volume of vehicle solution. Aside from a brief (5 to 10 sec) period of restlessness immediately following the injection, the mice displayed no obvious side effects from either the vehicle or SS injection. This procedure was repeated with fresh solutions on a daily basis for 68 (SS treated) and 70 (vehicle treated) consecutive days when each mouse received a final injection prior to being euthanized. The mice maintained their body weight and displayed no obvious side-effects to this chronic treatment schedule. After euthanizing the mice, the TS muscles were removed and intracellular recordings of resting potential were obtained using the techniques described in Carlson et al. (2005).

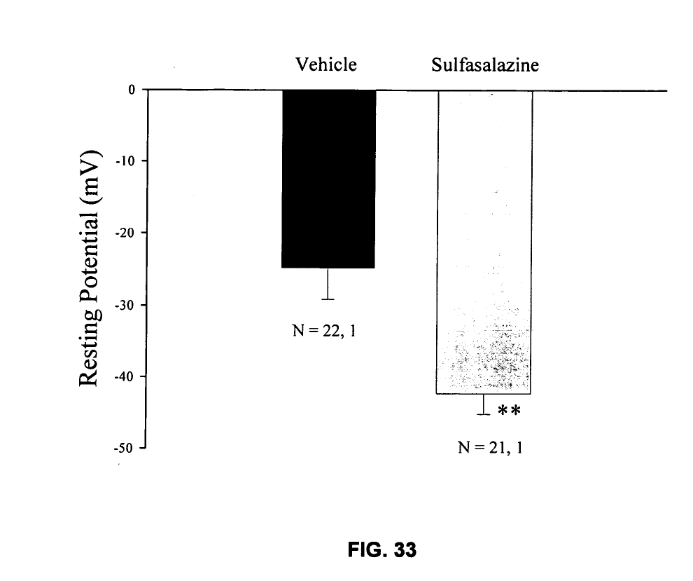

The results indicate that chronic treatment with SS significantly improved the resting potential of TS muscle fibers (FIG. 33) in a manner consistent with previous results obtained using chronic PDTC treatment (FIGS. 16, 17). The results indicate that chronic treatment with inhibitors of the NFκB pathway such as sulfasalazine have beneficial effects in treating muscular dystrophy (Carlson et al., 2005). Sulfasalazine had previously been shown to inhibit the NFκB pathway in a variety of human tissues (See Wahl et al., 1998; Gan et al., 2005; Lappas et al., 2005; and Robe et al. 2004). The results of electrophoretic shift assays (EMSA) following a single injection of SS provided the first demonstration that this drug effectively blocks the NFκB pathway in dystrophic muscle (FIG. 28). The available evidence indicates that sulfasalazine stabilizes IκB-α (Wahl et al., 1998) and inhibits IKK (Weber et al., 2000) in other tissues.

SS is used at similar doses in treatment of rheumatoid arthritis and ulcerative colitis. The results described here indicate that chronic treatment with SS has beneficial effects in improving the electrical characteristics of dystrophic muscle fibers by improving the resting membrane potential as initially demonstrated using the IκB-α stabilizing agent, PDTC (Carlson et al., 2005). These results also provide the first evidence that this drug may reduce nuclear NFκB activation in dystrophic muscle (FIG. 28) and that chronic treatment may substantially improve the electrical characteristics of dystrophic muscle fibers (FIG. 33). These results provide the first indication supporting the use of sulfasalazine in human clinical trials to treat Duchenne and related muscular dystrophies.

### REFERENCES

Almawi, W. Y. and O. K. Melemedjian, Negative regulation of nuclear factor-κB activation and function by glucocorticoids, J. Mol. Endocrinol. 28 (2002), pp. 69-78.
Andrade, F. H., J. D. Porter and H. J. Kaminski, Eye muscle sparing by the muscular dystrophies: lessons to be learned?, Microsc. Res. Tech. 48 (2000), pp. 192-203.
Arahata, K., S. Ishiura, T. Ishiguro, T. Tsukahara, Y. Suhara, C. Eguchi, T. Ishihara, I. Nonaka, E. Ozawa and H. Sugita, Immunostaining of skeletal and cardiac muscle surface membrane with antibody against Duchenne muscular dystrophy peptide, Nature 333 (1988), pp. 861-863.
Araki, S. and S. Mawatari, Ouabain and erythrocyte-ghost adenosine triphosphatase, Arch. Neurol. 24 (1971), pp. 187-190.
Ariga, A., J.-I. Namakawa, N. Matsumoto, J.-I. Inoue and K. Umezawa, Inhibition of tumor necrosis factor α-induced nuclear translocation and activation of NFκB by dehydroxymethylepoxyquinomicin, J. Biol. Chem. 277 (2002) (27), pp. 24625-24630.
Barnes, P. J., Molecules in focus: nuclear factor-κB, Int. J. Biochem. Cell Biol. 29 (1997) (6), pp. 867-870.
Blake, D. J., A. Weir, S. E. Newey and K. E. Davies, Function and genetics of dystrophin and dystrophin-related proteins in muscle, Physiol. Rev. 82 (2001), pp. 291-329.
Blondeau, N., C. Widmann, M. Lazdunski and C. Heurteaux, Activation of nuclear factor-κB is a key event in brain tolerance, J. Neurosci. 21 (2001) (13), pp. 4668-4677.
Broad, L. M., T. R. Cannon and C. W. Taylor, A non-capacitative pathway activated by arachidonic acid is the major Ca2+ entry mechanism in rat A7r5 smooth muscle cells stimulated with low concentrations of vasopressin, J. Physiol. 517 (1999), pp. 121-134.
Bulfield, G., W. G. Siller, P. A. L. Wright and K. J. Moore, X-chromosome-linked muscular dystrophy (mdx) in the mouse, Proc. Natl. Acad. Sci. U.S.A. 81 (1984), pp. 1189-1192.
Cai, D., J. D. Frantz, N. E. Tawa Jr., P. A. Melendez, B.-C. Oh, H. G. W. Lidov, P.-O. Hasselgren, W. R. Frontera, J. Lee, D. J. Glass and S. E. Shoelson, IKKβ/NF-κB activation causes severe muscle wasting in mice, Cell 119 (2004), pp. 285-298.
Carlson, C. G., The dystrophinopathies: an alternative to the structural hypothesis, Neurobiol. Dis. 5 (1998), pp. 3-15.
Carlson, C. G. and T. Geisbuhler, Diastolic calcium influx in isolated rat cardiac myocytes, Abstracts from the XXVth Annual Meeting, North American Section of the International Society for Heart Research, J. Mol. Cell. Cardiol. vol. 35 (6) (2003).
Carlson, C. G. and R. V. Makiejus. “A noninvasive procedure to detect muscle weakness in the mdx mouse.” Muscle and Nerve (1990) 13:480-484.
Carlson, C. G. and D. Roshek, Adult dystrophic (mdx) endplates exhibit reduced quantal sensitivity and enhanced quantal variation, Pflugers Arch. (Eur. J. Physiol.) 442 (2001), pp. 369-375.
Carlson, C. G., R. Ashmore, A. Gueorguiev, D. Roshek and J. Chu, Manganese quench rates and steady state calcium concentrations in cultured dystrophic myotubes and freshly excised adult dystrophic fibers, Neurosci. Abstr. vol. 27 (2001) Program number 105.14.
Carlson, C. G., A. Gueorguiev, D. M. Roshek, R. Ashmore, J. S. Chu and J. E. Anderson, Extra-junctional resting Ca2+ influx is not increased in a severely dystrophic expiratory muscle (triangularis sterni) of the mdx mouse, Neurobiol. Dis. 14 (2003) (2), pp. 229-239.
Chakkalakal, J. V., M.-A. Harrison, S. Carbonetto, E. Chin, R. N. Michel and B. J. Jasmin, Stimulation of calcineurin signaling attenuates the dystrophic pathology in mdx mice, Hum. Mol. Genet. 13 (2004) (4), pp. 379-388.
Cox, J. L., T. Lancaster and C. G. Carlson, Changes in the motility of B16F10 melanoma cells induced by alterations in resting calcium influx, Melanoma Res. 12 (2002), pp. 211-219.
Cullen, M. J. and J. J. Fulthorpe, Stages in fibre breakdown in Duchenne muscular dystrophy: an electron-microscopic study, J. Neurol. Sci. 24 (1975), pp. 179-200.
Cuzzocrea, S., P. K. Chatterjee, E. Mazzon, L. Dugo, I. Serraino, D. Britti, G. Mazzullo, A. P. Caputi and C. Thiemermann, Pyrrolidine dithiocarbamate attenuates the development of acute and chronic inflammation, Br. J. Pharmacol. 135 (2002), pp. 496-510.
D'Acquisto, F. D., A. Ialenti, T. Iuvone, M. DiRosa and R. Carnuccio, Inhibition of nuclear factor-κB prevents the loss of vascular tone in lipopolysaccharide-treated rats, Eur. J. Pharmacol. 365 (1999), pp. 253-257.
D'Acquisto, F. D., A. Ianaro, A. Ialenti, P. Maffia, M. C. Maiuri and R. Carnuccio, Transcription factor decoy oligodeoxynucleotides to nuclear factor-κB inhibit reverse passive Arthus reaction in rat, Naunyn-Schmiedeberg's Arch. Pharmacol. 364 (2001), pp. 422-429.
DeBacker, F., C. Vandebrouk, P. Gailly and J. M. Gillis, Long-term study of Ca2+ homeostasis and of survival in collagenase-isolated muscle fibres from normal and mdx mice, J. Physiol. (London) 542.3 (2002), pp. 855-865.
DeBosscher, K., W. V. Berghe and G. Haegeman, The interplay between the glucocorticoid receptor and nuclear factor-κB or activator protein-1: molecular mechanisms for gene repression, Endocr. Rev. 24 (2003) (4), pp. 488-522.
DeTroyer, A. and A. Legrand, Mechanical advantage of the canine triangularis sterni, J. Appl. Physiol. 84 (1998) (2), pp. 562-568.
DeTroyer, A. and V. Ninane, Triangularis sterni: a primary muscle of breathing in the dog, J. Appl. Physiol. 60 (1986), pp. 14-21.
DeTroyer, A., A. Legrand, P.-A. Gevenois and T. A. Wilson, Mechanical advantage of the human parasternal intercostal and triangularis sterni muscles, J. Physiol. (London) 513.3 (1998), pp. 915-925.
El-Bohy, A. A. and B. L. Wong, The diagnosis of muscular dystrophy, Pediatr Ann. (2005) 34(7):525-30.
Ervasti, J. M. and K. P. Campbell, Membrane organization of the dystrophin-glycoprotein complex, Cell 66 (1991), pp. 1121-1131.
Fenichel, G. M., J. M. Florence, A. Pestronk, J. R Mendell, R. T. Moxley III, R. C. Griggs, M. H. Brooke, J. P. Miller, J. Robison, W. King, L. Signore, S. Pandya, J. Schierbecker and B. Wilson, Long-term benefit from prednisone therapy in Duchenne muscular dystrophy, Neurology 41 (1991), pp. 1874-1877.
Franco Jr., A., B. D. Winegar and J. B. Lansman, Open channel block by gadolinium ion of the stretch-inactivated ion channel in mdx myotubes, Biophys. J. 59 (1991), pp. 1164-1170.
Gan et al., J. of Gastroenterology and Hepatology, (2005) 20:1016-24.
Gosselin, L. C., J. E. Barkley, M. J. Spencer, K. M. McCormick and G. A. Farkas, Ventilatory dysfunction in mdx mice: impact of tumor necrosis factor-alpha deletion, Muscle Nerve 28 (2003), pp. 336-343.
Guttridge, D. C., M. W. Mayo, L. V. Madrid, C.-Y. Wang and A. S. Baldwin Jr., NF-κB-induced loss of Myo D messenger RNA: a possible role in muscle decay and cachexia, Science 289 (2000), pp. 2363-2366.
Hoffman, E. P., R. H. Brown and L. M. Kunkel, Dystrophin: the protein product of the Duchenne muscular dystrophy locus, Cell 51 (1987), pp. 919-928.
Hwang, J.-C., D. Zhou and W. M. St. John, Characterization of expiratory intercostal activity to triangularis sterni in cats, J. Appl. Physiol. 67 (1989) (4), pp. 1518-1524.
Jimi, E., K. Aoki, H. Saito, F. D. D'Acquisto, M. J. May, I. Nakamura, T. Sudo, T. Kojima, F. Okamoto, H. Fukushima, K. Okabe, K. Ohya and S. Ghosh, Selective inhibition of NF-κB blocks osteoclastogenesis and prevents inflammatory bone destruction in vivo, Nat. Med. 10 (2004) (6), pp. 617-624.
Jonsdottir, I. H., P. Scherling, K. Ostrowski, S. Asp, E. A. Richter and B. K. Pedersen, Muscle contractions induce interleukin-6 mRNA production in rat skeletal muscles, J. Physiol. 528.1 (2000), pp. 157-163.
Karin, M., Y. Yamamoto and Q. M. Wang, The IKK NF-κB system: a treasure trove for drug development, Nat. Rev. 3 (2004), pp. 17-26.
Koenig, M. and L. M. Kunkel, Detailed analysis of the repeat domain of dystrophin reveals four potential hinge segments that may confer flexibility, J. Biol. Chem. 265 (1990), pp. 4560-4566.
Kreydiyyeh, S. I. and R. Al-Sadi, Interleukin-1 beta increases urine flow rate and inhibits protein expression of Na+/K+ ATPase in the rat jejunum and kidney, J. Interferon Cytokine Res. 22 (2002) (10), pp. 1041-1048.
Kreydiyyeh, S. I., C. Abou-Chahine and R. Hilal-Dandan, Interleukin-1 beta inhibits Na+-K+ ATPase activity and protein expression in cardiac myocytes, Cytokine 26 (2004) (1), pp. 1-8.
Kumar, A. and A. M. Boriek, Mechanical stress activates the nuclear factor-kappa B pathway in skeletal muscle fibers: a possible role in Duchenne muscular dystrophy, FASEB J. 17 (2003), pp. 296-386.
Lang, C. H., C. Silvis, N. Deshpande, G. Nystrom and R. A. Frost, Endotoxin stimulates in vivo expression of inflammatory cytokines tumor necrosis factor alpha, interleukin-1beta,-6, and high mobility-group protein-1 in skeletal muscle, Shock 19 (2003) (6), pp. 538-546.
Lappas et al., Endocrinology, (2005) 146 (3), 1491-1497.
Li, X., P. E. Massa, A. Hanidu, G. W. Peet, P. Aro, A. Savitt, S. Mische, J. Li and K. B. Marcu, IKKα, IKKβ, and NEMO/IKKγ are each required for the NF-κB-mediated inflammatory response program, J. Biol. Chem. 277 (2002) (47), pp. 45129-45140.
Lloyd, S. J. and A. E. H. Emery, A possible circulating plasma factor in Duchenne muscular dystrophy, Clin. Chim. Acta 112 (1981), pp. 85-90.
Matsuda, R., A. Nishikawa and H. Tanaka, Visualization of dystrophic muscle fibers in mdx mouse by vital staining with Evans blue: evidence of apoptosis in dystrophin-deficient muscle, J. Biochem. 118 (1995), pp. 959-964.
Matsumura, K. and K. P. Campbell, Dystrophin-glycoprotein complex: its role in the molecular pathogenesis of muscular dystrophies, Muscle Nerve 17 (1994), pp. 2-15.
Monici, M. C., M. Aguennouz, A. Mazzeo, C. Messina and G. Vita, Activation of nuclear factor-κB in inflammatory myopathies and Duchenne muscular dystrophy, Neurology 60 (2003), pp. 993-997.
Nagel, A., F. Lehmann-Horn and A. G. Engel, Neuromuscular transmission in the mdx mouse, Muscle Nerve 13 (1990), pp. 742-749.
Ninane, V., R. E. Baer and A. De Troyer, Mechanism of triangularis sterni shortening during expiration in dogs, J. Appl. Physiol. 66 (1989) (5), pp. 2287-2292.
Orlowski, R. Z. and A. S. Baldwin Jr., NF-κB as a therapeutic target in cancer, Trends Mol. Med. 8 (2002) (8), pp. 385-389.
Peter, J. B., M. Worsfold and C. M. Pearson, Erythrocyte ghost adenosine triphosphatase (ATPase) in Duchenne dystrophy, J. Lab. Clin. Med. 74 (1969) (1), pp. 103-108.
Petrof, B. J., J. B. Shrager, H. H. Stedman, A. M. Kelly and H. L. Sweeney, Dystrophin protects the sarcolemma from stresses developed during muscle contraction, Proc. Natl. Acad. Sci. U.S.A. 90 (1993), pp. 3710-3714.
Rangan, G. K., Y. Wang, Y.-C. Tay and D. C. H. Harris, Inhibition of nuclear factor-κB activation reduces cortical tubulointerstitial injury in proteinuric rats, Kidney Int. 56 (1999), pp. 118-134.
Robe et al., Clinical Cancer Research, (2004)10, 5595-5603.
Sakakibara, H., A. G. Engel and E. H. Lambert, Duchenne dystrophy: ultrastructural localization of the acetylcholine receptor and intracellular microelectrode studies of neuromuscular transmission, Neurology 27 (1977), pp. 741-745.
Samadi, A., R. J. Cenedella and C. G. Carlson, Diethylstillbestrol (DES) increases intracellular calcium in lens epithelial cells, Pfluegers Arch. (Eur. J. Physiol.) (2005).
Satoh, A., T. Shimosegewa, M. Fujita, A. Masamune, M. Koizumi and T. Toyota, Inhibition of nuclear factor-κB activation improves the survival of rats with taurocholate pancreatitis, Gut 44 (1999), pp. 253-258.
Siebenlist, U., G. Franzoso and K. Brown, Structure, regulation and function of NF-κB, Annu. Rev. Cell Biol. 10 (1994), pp. 405-455.
Singh, S. and B. B. Aggarwal, Activation of transcription factor NF-κB is suppressed by curcumin (diferulolylmethane), J. Biol. Chem. 270 (1995) (42), pp. 24995-25000.
Takeuchi, T., S. Miura, L. Wang, K. Uehara, M. Mizumori, H. Kishikawa, R. Hokari, H. Higuchi, M. Adachi, H. Nakamizo and H. Ishii, Nuclear factor-κB and TNF-α mediate gastric ulceration induced by phorbol myristate acetate, Dig. Dis. Sci. 47 (2002), pp. 2070-2078.
Thaloor, D., K. J. Miller, J. Gephart, P. O. Mitchell and G. K. Pavlath, Systemic administration of the NF-κB inhibitor curcumin stimulates muscle regeneration after traumatic injury, Am. J. Physiol.: Cell Physiol. 46 (1999), pp. C320-C329.
Tutdibi, O., H. Brinkmeir, R. Rudel and K. J. Fohr, Increased calcium entry into dystrophin-deficient muscle fibers of MDX and ADR-MDX mice is reduced by ion channel blockers, J. Physiol. (London) 515.3 (1999), pp. 859-868.
Wahl et al., J. Clin. Invest., (1998) 101, 1163-74.
Weber et al., Gastroenterology, (2000) 119, 1209-18.
Weller, B., G. Karpati and S. Carpenter, Dystrophin-deficient mdx muscle fibers are preferentially vulnerable to necrosis induced by experimental lengthening contractions, J. Neurol. Sci. 100 (1990), pp. 9-13.
Yang, X. C. and F. Sachs, Block of stretch-activated ion channels in Xenopus oocytes by gadolinium and calcium ions, Science 243 (1989), pp. 1068-1071.
Yeung, E. W., S. I. Head and D. G. Allen, Gadolinium reduces short-term stretch-induced muscle damage in isolated mdx mouse muscle fibres, J. Physiol. (London) 552.2 (2003), pp. 449-458.
Yuksel, M., K. Okajima, M. Uchiba and H. Okabe, Gabaxate mesilate, a synthetic protease inhibitor, inhibits lipopolysaccharide-induced tumor necrosis factor-α production by inhibiting activation of both nuclear factor-κB and activator protein-1 in human monocytes, J. Pharmacol. Exp. Ther. 305 (2003), pp. 298-305.

We claim:

1. A method for treating muscular dystrophy in a subject comprising the step of administering to the subject an agent in an effective amount which decreases the level or the activity of NFκB in the muscular tissues of the subject.

2. The method of claim 1 wherein the agent is an NFκB inhibitor.

3. The method of claim 2 wherein the NFκB inhibitor is selected from the group consisting of pyrrolidine dithiocarbamate; curcumin; SN-50; gabaexate mesilate; BMS-345541; a quinazoline analogue identified as SPC-839, a beta-carbolin analogue identified as PS-1145; an amino-thiophenecarboxamide derivative identified as SC-514; ureido-thiophenecarboxamide derivatives diarylpyridine derivatives; anilino-pyrimidine derivatives; pyridooxazinone derivatives; indolecarboxamide derivatives; benzoimidazole carboxamide derivatives; pyrazolo(4,3-c) quinoline derivatives; imidazolylquinoline-carboxaldehyde semicarbazide derivatives; amino-imidazolecarboxamide derivatives; pyridyl cyanoguanidine derivatives; epigallocatechin-3-gallate and similar polyphenols extracted from green tea, diethyldithiocarbamate; κB decoy DNA sequences; MG 132; a peptide Leu-Asp-Trp-Ser-Trp-Leu; 3,4-dichloropropionaniline; water soluble extract of Uncaria tomentosa termed C-Med 100; hydro-alcoholic extract of Uncaria tomentosa; dehydroxymethylepoxyquinomicin, pirfenidone (2(1H)-pyridinone 5-methyl-1-phenyl); Bay 11-7085; Bay 11-7082; gliotoxin; parthenolide; artemisinin; helenalin; mexicanin I; 2,3-dihydroaromaticin; helenalin-isobutyrate; isohelenin; arctigenin and related dibenzylbutyrolactone lignans such as demethyltraxillagenin;sulfasalazine; guggelsterone; troglitazone; methanol extract of the plant Saururus chinensis; N-acetylcysteine; phenylmethyl benzoquinone derivatives; xanthine derivatives; isoquinoline derivatives; indan derivatives; alkaloids originated from a plant belonging to the genus Stephania of the family Menspermaceae; peptides including the recognition domain for E3 ubiquitin ligase; antisense oligonucleotides which hybridize to NF-κB mRNA and thus inhibit NF-κB dependent pathways; and combinations thereof

4. The method of claim 1 wherein the agent is administered as a pharmaceutical composition.

5. The method of claim 1 further comprising the step of monitoring NFκB levels in the subject to ascertain the effect of treatment.

6. A method for treating muscular dystrophy comprising administering to a subject diagnosed with muscular dystrophy a pharmaceutical composition comprising an inhibitor of NFκB activation in an amount effective to improve the whole body strength of the subject.

7. The method of claim 6 further comprising the step of monitoring the whole body strength of the subject to ascertain the effect of treatment.

8. The method of claim 6 further comprising the step of monitoring the tension generated by functionally isolated limb musculature of the subject to ascertain the effect of treatment.

9. A method for treating muscular dystrophy comprising the steps of:
(a) diagnosing a subject in need of treatment for muscular dystrophy;
(b) administering to said subject an inhibitor of NFκB activation in an amount effective to inhibit activation of NFκB in said subject; and
(c) permitting the inhibitor to achieve therapeutic benefit for muscular dystrophy in said subject.

10. A method for treating muscular dystrophy comprising administering to a subject diagnosed with muscular dystrophy a pharmaceutical composition comprising an inhibitor of NFκB activation on a chronic basis.

11. The method of claim 10 wherein the inhibitor of NFκB activation is administered in an amount effective to substantially reduce the total cellular levels of NFkappaB in isolated dystrophic skeletal muscle.

12. The method of claim 10 wherein the inhibitor of NFκB activation is administered in an amount effective to substantially reduce the percentage of total cellular NFkappaB that is localized to the nuclear compartment of isolated dystrophic skeletal muscle.

13. The method of claim 10 wherein the inhibitor of NFκB activation is administered in an amount effective to substantially improve the resting electrical properties and resting membrane potential of isolated dystrophic skeletal muscle fibers in the subject.

14. The method of claim 10 wherein the inhibitor of NFκB activation is administered in an amount effective to increase the number of surviving striated muscle fibers in isolated skeletal muscles that are subjected to passive stretch during normal use.

15. The method of claim 10 wherein the inhibitor of NFκB activation is administered in an amount effective to increase the total number of muscle fibers in skeletal muscles that are subjected to passive stretch during normal use.

16. The method of claim 10 wherein the inhibitor of NFκB activation is administered in an amount effective to increase the number of skeletal muscle nuclei per muscle fiber in skeletal muscles that are subjected to passive stretch during normal use.

17. The method of claim 10 wherein the inhibitor of NFκB activation is administered in an amount effective to increase the cross-sectional area of individual dystrophic muscle fibers in certain regions of skeletal muscle fibers that are subjected to passive stretch during normal use.

18. The method of claim 10 wherein the inhibitor of NFκB activation is administered in an amount sufficient to reduce percent centronucleation.

19. The method of claim 10 wherein the inhibitor of NFκB activation is administered in an amount effective to improve the total tension generated by isolated muscles in the limbs of dystrophic subjects.

20. The method of claim 10 wherein the inhibitor of NFκB activation is administered in an amount effective to improve the whole body strength of dystrophic subjects.

21. A method for treating muscular dystrophy comprising the step of administering a chemical or biological agent to a subject to inhibit the expression of at least one NFκB-dependent cytokine.

22. The method of claim 21 wherein the NFκB dependent cytokine is selected from the group consisting of IL-1β, IL-6 and TNFα or combination thereof.

23. The method of claim 21 further comprising the step of monitoring the levels of the at least one NFκB-dependent cytokine in the skeletal muscle of said subject.

24. The method of claim 23 wherein the monitoring is through monitoring the gene expression profile.

25. The method of claim 23 wherein the monitoring is through immunoassay.

26. A method for treating muscular dystrophy comprising:
(a) administering to a subject diagnosed with muscular dystrophy a pharmaceutical composition comprising an inhibitor of NFκB activation in an amount effective to increase the total number of muscle fibers in skeletal muscles that are subjected to passive stretch during normal use; and
(b) monitoring the total number of muscle fibers in skeletal muscles of the subject that are subjected to passive stretch during normal use, in order to ascertain the effect of treatment.

27. A method for treating muscular dystrophy comprising:
(a) administering to a subject an inhibitor of NFκB activation in an amount that is effective to inhibit NFκB activation in the muscle cells of the subject, the inhibitor of NFκB activation being capable of reducing NFκB activation at a predetermined first level; and
(b) administering to the subject at least one additional inhibitor of NFκB activation in an amount that is effective to inhibit NFκB activation in the muscle cells of said subject, said additional inhibitor of NFκB activation being capable of reducing NFκB activation at a predetermined second level that is different from the predetermined first level.

28. A method for treating muscular dystrophy comprising:
(a) administering to a subject an inhibitor of NFκB activation in a first amount that is effective to inhibit NFκB activation in the muscle cells of the subject, the inhibitor of NFκB activation being capable of reducing NFκB activation at a predetermined first level; and
(b) administering to the subject said inhibitor of NFκB activation in a second amount that is effective to inhibit NFκB activation in the muscle cells of said subject, said second amount of inhibitor of NFκB activation being capable of reducing NFκB activation at a predetermined second level that is different from the predetermined first level.

29. A composition for use in the treatment of muscular dystrophy, comprising:
(a) an inhibitor of NFκB activation in an amount that is effective to inhibit NFκB activation in the muscle cells of a subject, the inhibitor of NFκB activation being capable of reducing NFκB activation at a predetermined first level; and
(b) at least one additional inhibitor of NFκB activation in an amount that is effective to inhibit NFκB activation in the muscle cells of said subject, said additional inhibitor of NFκB activation being capable of reducing NFκB activation at a predetermined second level that is different from the predetermined first level.
